(19) United States
(12) Patent Application Publication
(10) Pub. No.: US 2010/0272698 A1
(43) Pub. Date: Oct. 28, 2010

(54) YEASTS

(76) Inventors: Lubomira Stateva, Manchester (GB); Steven George Oliver, Cambridge (GB); Walid Omara, Manchester (GB)

Correspondence Address: BOSE MCKINNEY & EVANS LLP, 111 MONUMENT CIRCLE, SUITE 2700, INDIANAPOLIS, IN, 46204, US

(21) Appl. No.: 12/709,700

(22) Filed: Feb. 22, 2010

Related U.S. Application Data
(60) Provisional application No. 61/154,521, filed on Feb. 23, 2009.

(51) Int. Cl. A61K 36/06; C12N 1/19; C12N 15/81

(52) U.S. Cl. 424/93.21; 435/254.2; 435/320.1; 435/471

(57) ABSTRACT

A yeast cell is provided comprising an artificial nucleic acid construct incorporating a gene associated with yeast cell wall integrity operatively linked to a promoter responsive to conditions characteristic of the digestive tract. Also provided is a method comprising administering to the digestive tract of the subject a yeast cell comprising an artificial nucleic acid construct comprising a gene associated with yeast cell wall integrity, operatively linked to a promoter responsive to conditions characteristic of the digestive tract, wherein the yeast cell further comprises an amount of an agent of interest encapsulated within said yeast cell.

### BACKGROUND

The present invention relates to yeast cells, to pharmaceutical compositions comprising such yeast cells, and to therapeutic uses of the yeast cells and/or pharmaceutical compositions. The invention also relates to artificial nucleic acid constructs useful in such yeast cells, and the use of such artificial nucleic acid constructs to produce yeast cells adapted for targeted lysis in the digestive tract.

Therapeutically effective peptides have utility in the prevention and/or treatment of a wide range of disease or other adverse conditions. These are now well studied and accepted by regulatory authorities, from early uses of insulin to treat diabetes, and vaccines to induce immunity, to more recent therapeutic applications of cytokines and other soluble factors. Increasing understanding of the proteins encoded by the genome, as well as the links between protein structure and function, has allowed the range of naturally occurring therapeutic proteins to be augmented by modified or artificial compounds.

In order to produce such therapeutic molecules on a commercial scale it has been necessary to generate these molecules by means other than their natural cellular sources. Sources that have frequently been used include mammalian cell cultures, as well as micro-organisms such as bacteria or yeasts.

As an expression system, yeast provides many advantages because proteins produced by the yeast system are likely to be produced with their normal eukaryotic folding and post-translational modifications. The yeast culture is also easier to use and maintain than other higher eukaryote expression systems. Yeast-based expression systems, as well as being simple, easy to modify and scale up to commercially relevant production, also benefit from a sophisticated intracellular transport and protein maintenance system that is similar to that found in vertebrate cells. In contrast to bacteria, well characterised yeast species such as S. cerevisiae provide model eukaryotic expression systems which are ideally suited for the expression of human and animal peptide drug candidates.

Peptide therapeutic agents are subject to degradation by a range of chemical conditions, and particularly those found in biological systems. Accordingly, the delivery of peptide-based therapies has commonly been achieved via intravenous, intramuscular or subcutaneous injection. However these routes of administration have a low patient compliance, particularly in the fields of paediatric and geriatric medicine where ease-of-use of drugs is vital. Injections are also invasive; require a level of sterility and present risk of infection, in what may already be immunocompromised patients.

In the light of these well-recognised disadvantages, there is a need to provide alternative means by which therapeutically effective agents, such as peptides, may be administered to patients in a form in which they may exert their therapeutic activity.

It has long been recognised that it would be desirable to identify a means by which such therapies may be delivered orally. This method of drug delivery has the highest level of patient compliance, avoids pain and discomfort, eliminates infection risk and thus increases the therapeutic value of a drug. However, the conditions found in the digestive tract are adapted for the breakdown of peptides (whether by enzyme activity or the chemical conditions of extreme pH or temperature) in keeping with the use of proteins as food sources.

Recent attempts to use living recombinant microorganisms, to deliver active compounds via the oral route, have provided a new strategy for the prevention or treatment of disease. However, the practical use of these approaches has been markedly limited by the inability of the engineered vehicles to achieve biologically significant levels of secretion of the proteins expressed, unless limited to very small therapeutic peptides. Furthermore, concern has been expressed regarding the risk that populations of such genetically modified micro-organisms resident in a host may give rise to opportunistic infections, particularly in patient groups such as the young, elderly or immunocompromised. Such approaches also suffer from the disadvantage that there is no control over concentrations of the microorganisms produced within the host, or of the duration of time the delivery cells are alive and functional in the host's gut. Furthermore, in order to minimise the dissemination and release of genetically modified microorganisms of this sort into the environment (via excretion) sophisticated systems able to ensure the controlled death of the cells outside the host must be implemented.

Genetically engineered yeast strains have been produced that, upon exposure to tightly controlled concentrations of extracellular factors such as methionine and cysteine, are induced to undergo cell lysis, through down-regulated expression of essential genes encoding yeast cell wall constituents. It has been reported that the induction of lysis in such yeast cells requires at least 8 hours incubation under conditions able to influence gene expression and thus reduce cell wall integrity. Compounds such as methionine and cysteine are well known for their ability to influence the activity of some gene promoters in this manner, but, in order to exert their activity, must be provided to the yeast cells at concentrations not found in naturally occurring biological conditions. Accordingly, these genetically engineered yeasts of the prior art are not suitable for use in the delivery of agents of interest (such as therapeutic agents) to a subject. Examples of such yeast strains are disclosed in the inventors' earlier U.S. Pat. No. 6,800,476, the disclosure of which is herein incorporated by reference.

### SUMMARY OF THE INVENTION

In one aspect of the present invention there is provided a yeast cell comprising an artificial nucleic acid construct incorporating a gene associated with yeast cell wall integrity operatively linked to a promoter responsive to conditions characteristic of the digestive tract.

In another aspect of the present invention there is provided a method of providing an agent of interest to a subject in need thereof, the method comprising administering to the digestive tract of the subject a yeast cell comprising an artificial nucleic acid construct comprising a gene associated with yeast cell wall integrity, operatively linked to a promoter responsive to conditions characteristic of the digestive tract, wherein the yeast cell further comprises an amount of an agent of interest encapsulated within said yeast cell.

In a further aspect of the present invention there is provided a pharmaceutical composition comprising a yeast cell incorporating an artificial nucleic acid construct incorporating a gene associated with yeast cell wall integrity operatively linked to a promoter responsive to conditions characteristic of the digestive tract.

In yet another aspect of the present invention there is provided an artificial nucleic acid construct comprising a gene associated with yeast cell wall integrity operatively linked to a promoter responsive to conditions characteristic of the digestive tract.

In a further aspect of the present invention there is provided a method of producing a yeast cell adapted for targeted lysis in the digestive tract, the method comprising introducing an artificial nucleic acid construct, incorporating a gene associated with yeast cell wall integrity operatively linked to a promoter responsive to conditions characteristic of the digestive tract, into a yeast cell, such that the artificial nucleic acid construct is expressible in the yeast cell.

### DETAILED DESCRIPTION

It is an aim of certain embodiments of the present invention to obviate or mitigate at least some of the shortcomings of the prior art. It is an aim of certain embodiments of the present invention to provide improved vehicles for the provision of agents of interest to a subject. It is an aim of certain embodiments of the present invention to provide improved vehicles for the provision of therapeutic agents of interest to a subject. It is an aim of certain embodiments of the present invention to provide methods for the provision of therapeutic agents to a subject.

In a first aspect, the present invention provides a yeast cell comprising an artificial nucleic acid construct incorporating a gene associated with yeast cell wall integrity operatively linked to a promoter responsive to conditions characteristic of the digestive tract. Since the nucleic acid construct is artificial, it will be appreciated that the gene associated with yeast cell wall integrity is thus operatively linked to a promoter that does not regulate its expression in naturally occurring yeast cells. The promoters and/or genes may be heterologous or homologous, though it may generally be preferred to use homologous promoters and/or genes.

In a second aspect, the invention provides a pharmaceutical composition comprising a yeast cell comprising an artificial nucleic acid construct incorporating a gene associated with yeast cell wall integrity operatively linked to a promoter responsive to conditions characteristic of the digestive tract. The pharmaceutical composition may optionally comprise a pharmaceutically acceptable excipient or diluent. Pharmaceutical compositions in accordance with the invention may be of solid or liquid form.

In a third aspect, the invention provides an artificial nucleic acid construct comprising a gene associated with yeast cell wall integrity operatively linked to a promoter responsive to conditions characteristic of the digestive tract. As in the yeasts of the invention, the promoter is one that does not naturally regulate the expression of the gene associated with yeast cell wall integrity. Except for where the context requires otherwise, the various embodiments described herein with reference to yeast cells of the invention will also be applicable to nucleic acid constructs in accordance with the third aspect of the invention.

In a fourth aspect, the invention provides a method of producing a yeast cell adapted for targeted lysis in the digestive tract, the method comprising introducing an artificial nucleic acid construct, incorporating a gene associated with yeast cell wall integrity operatively linked to a promoter responsive to conditions characteristic of the digestive tract, into a yeast cell, such that the artificial nucleic acid construct is expressible in the yeast cell.

The present invention makes use of the inventors' surprising finding that yeasts possess genes whose transcript abundances within yeast cells alter dramatically in response to conditions found in the digestive tract, and that the promoters of these genes (which will generally be referred to herein simply as “promoters”, for the sake of brevity) may be used to control expression of genes associated with yeast cell wall integrity such that targeted lysis of the yeast cells within the digestive tract may be achieved. Thus it is possible to produce yeast cells in accordance with the present invention that will undergo lysis at a targeted site where it is wished to provide an agent of interest (the resilient cell wall of unaltered yeast cells usually providing sufficient to prevent cell lysis within the digestive tract) for example, the small intestine or other sites where agent of interest may be absorbed. The ability to induce targeted lysis of the yeast cells of the invention can also be used to ensure that agents of interest are not released at sites where they may otherwise be subject to digestion or other degradation and thus prevented from exerting their desired activity.

The digestive tract-responsive promoters identified in the present specification were found as part of the first study intended to identify yeast genes that vary their expression in response to conditions found in the digestive system. The conditions found in the digestive tract are of such complexity that it has previously proven difficult to predict how they will influence gene expression, and such predictions have been of little practical value.

The genes found to have promoters that respond to conditions characteristic of the digestive tract fulfil a wide range of biological functions, indicating that there were multiple cellular responses involved. Surprisingly, the inventors found that few of these genes were associated with the yeast cells' response to stress caused by the conditions in the digestive tract.

The inventors have identified populations of genes that alter their expression in response to conditions found in all digestive tract compartments investigated, and also genes that alter their expression in response to conditions found in some digestive tract compartments, but not in others. The applications of these different types of promoters are described further below.

Now that the inventors have identified such genes, the present invention is based upon the use of their promoters to control other genes of interest, such as those associated with yeast cell wall integrity. The identification of these gene promoters obviates the need to use “external” inducers or repressors of gene expression. Instead, desired patterns of gene expression may be established in response to naturally occurring conditions, through operative linkage of a promoter with a desired expression profile (in terms of its up-regulation or down-regulation of expression in response to conditions in a digestive tract site of interest) to a gene encoding a product having a desired activity.

In the present case, these promoters are incorporated in an artificial nucleic acid construct, in which they are operatively linked to a gene associated with yeast cell wall integrity. Thus these constructs may be used to induce lysis of yeast cells of the invention in response to selected conditions characteristic of the digestive tract. Once the yeast cells lyse their cytoplasmic contents, including any intracellular proteins of interest, will be released into the digestive tract.

The promoters identified by the inventors may also be used to regulate the expression of such proteins of interest (including therapeutic agents) by the yeast cells. That said, it will generally be preferred that expression of such proteins is controlled by other yeast promoters known to have utility in heterologous expression systems, since these will facilitate accumulation of the protein of interest in the yeast cells prior to their administration to a patient. Examples of such promoters are considered elsewhere in the present disclosure.

It will be appreciated that yeast cells in accordance with the invention are able to act as highly effective vehicles for the delivery of therapeutic agents. The agents are encapsulated in the yeast cells, and the yeast cells orally administered to a subject requiring therapy. Once the yeast cells reach the selected compartment of the digestive tract they lyse, through the actions of the genes controlled by the responsive promoters, and in doing so release the therapeutic agent.

Yeast cells of the invention serve to preserve encapsulated agents of interest, such as peptide-based drugs and vaccines, and provide an effective means by which such agents may be non-invasively delivered. The encapsulated agents are protected from digestion after oral administration (for example in the harsh condition of the stomach), and also aided in their absorption at the mucosal surface of the gut.

Accordingly, in a fifth aspect of the invention there is provided a method of providing an agent of interest to a subject in need thereof, the method comprising administering to the digestive tract of the subject a yeast cell comprising an artificial nucleic acid construct comprising a gene associated with yeast cell wall integrity, operatively linked to a promoter responsive to conditions characteristic of the digestive tract, wherein the yeast cell further comprises an amount of an agent of interest encapsulated within said yeast cell. Exposure of the yeast cell to conditions characteristic of the digestive tract will then cause the yeast cell to lyse, and the agent of interest will thus be provided to the subject. It may be preferred that the agent of interest is a therapeutic agent able to prevent and/or treat a disease or condition detrimental to the subject.

Yeast cells of the invention are suitable for oral administration to a subject, and serve to protect encapsulated agents of interest from breakdown prior to cell lysis. This provides significant advantages, since it is known that oral drug delivery has the highest level of patient compliance, avoids pain and discomfort, eliminates infection risk and thus increases the therapeutic value of a drug, but that this route of administration had not previously been available for the majority of therapeutically effective peptides.

The yeast cells of the invention may be administered in solid form or in the form of a solution. They may be administered as part of a solid or liquid pharmaceutical composition. They may be administered as part of a foodstuff, as part of a beverage, or in tablet or other form conventional in the art.

It will generally be preferred that the therapeutic agents encapsulated within the yeast cells are expressed by the yeast cells themselves, and this embodiment is discussed in greater detail elsewhere in the specification.

Release of a therapeutic agent (or other protein of interest) through yeast cell lysis, as opposed to, for example, secretion by yeast cells, provides a number of advantages. There is less restriction on the size or nature of the therapeutic agents that can be used, since they need not be capable of, or adapted for, secretion through the yeast cell membrane and wall. It is also not necessary for the yeast cells to remain viable in the digestive tract for a prolonged period, which may otherwise be necessary if agents are to be expressed and secreted in situ. This provides a number of advantages, in that it reduces the likelihood of opportunistic infection by the yeast cells, and limits the requirement to identify and use yeast species able to survive in the inhospitable digestive tract environment. Furthermore induced lysis of the yeast cells in the digestive tract obviates the need for a system to minimise and abolish release of viable genetically modified organisms in the environment. As noted above, the protective cell walls of yeast cells mean that they are normally able to pass through the digestive tract substantially unharmed, and thus may be released into the environment by the normal process of excretion.

Delivery of agents of interest, such as therapeutic agents, via yeast cell lysis also allows for greater control of the amount of the agent delivered. A known number of yeast cells each containing a known quantity of the agent of interest may be provided to a subject. Thus the total dose provided is well regulated, even if the yeast cells undergo some degree of replication between administration and their subsequent lysis. This is in contrast to techniques in which a known number of cells that will then replicate over a prolonged period are provided to a subject, and these cells then produce and secrete an unspecified (and possibly uncontrolled) amount of an agent.

Suitable therapeutic agents that may be delivered using the yeast cells of the invention include therapeutically effective proteins capable of uptake through the digestive tract (to achieve their effect at other body sites), as well as those that will exert a direct therapeutic action on the digestive tract. Of particular utility are components associated with protein vaccines. Further details, and preferred embodiments, of these therapeutic agents are described in more detail below.

Various terms that are used in the present disclosure to describe the invention will now be explained further. The definitions and guidance provided below may be expanded on elsewhere in the specification as appropriate, and as the context requires.

### “Yeast Cells”

Yeast cells in accordance with the present invention may be derived from any suitable species of yeast. Merely by way of example, species of yeast that may be used to provide yeast cells in accordance with the present invention may be selected from the group consisting of: Saccharomyces, Kluyveromyces, Candida, Pichia, Schizosaccharomyces and Yarrowia. Various different strains of S. cerevisiae may be used including those utilised for baking, brewing, wine or probiotic applications. The probiotic strain S. boulardii may be of particular preference, as discussed further below; Other preferred species or strains include Saccharomyces carlsbergensis, Candida kefyr, Candida tropicalis, Kluyveromyces lactis, Kluyveromyces fragilis, Pichia pastoris, Schizosaccharomyces pombe, Hansenula polymorpha and Yarrowia lipolytica.

Suitable species or strains of yeast for use in accordance with the invention may be selected with reference to their abilities to express agents of interest, and/or their propensities to undergo targeted lysis and thereby release such agents. The various gene expression promoters described elsewhere in the present application are of particular relevance to the response of S. cerevisiae to conditions found in the digestive tract. Accordingly, in a preferred embodiment yeast cells of the invention may comprise, or be derived from, S. cerevisiae.

The inventors have found that the probiotic strain of S. cerevisiae S. boulardii exhibits a high tolerance for conditions found in the digestive tract (high tolerance to low pH in particular), and is capable of growth in the gastric compartment. Such properties are advantageous, since it will be appreciated that in order for the targeting of lysis to be effective, yeast cells in accordance with the invention should remain generally viable and intact until they reach the location in which it is desired for them to release their contents. Accordingly, in a particularly preferred embodiment yeast cells of the invention may comprise, or be derived preferably from, S. boulardii.

“Genes Associated with Yeast Cell Wall Integrity”

Yeast cell wall integrity is inversely linked to the propensity of a yeast cell to undergo lysis. Genes associated with yeast cell wall integrity that may be employed in the present invention are preferably those associated with the biogenesis and hence accumulation of cell wall components (in which case the gene is associated with yeast cell wall integrity in that it increases such integrity), or the degradation of cell wall components (in which case the gene is associated with yeast cell wall integrity in that it decreases such integrity).

Thus suitable genes associated with yeast cell wall integrity may be genes expression of which will increase accumulation of cell wall components. Examples of these will include genes encoding components or regulators of the synthesis of components the yeast cell wall. Such genes are associated with yeast cell wall integrity in that a reduction in their expression may be expected to compromise the cell wall integrity and thus increase the likelihood of yeast cell lysis.

It will also be appreciated that genes associated with yeast cell wall integrity may be genes expression of which increases the degradation of cell wall components. In this case, suitable genes may encode proteins, such as enzymes, that cause the degradation of yeast cell wall components. Genes of this sort are associated with yeast cell wall integrity in that their increased expression may be expected to decrease yeast cell wall integrity, and thereby promote yeast cell lysis.

Further examples of specific genes associated with yeast cell wall integrity, including both proteins associated with cell wall biogenesis and enzymes associated with cell wall degradation, are set out below.

It may be preferred that the yeast cells of the invention are modified such that expression of a gene associated with yeast cell wall integrity is permanently altered, in addition to the alteration of gene expression in response to conditions characteristic of the digestive tract. The permanent alteration of gene expression in this manner may help predispose the yeast cells to lysis, and thereby reduce the time for which it is necessary for them to be exposed to digestive tract conditions prior to targeted lysis (under the influence of promoters responsive to conditions characteristic of the digestive tract) occurring.

By way of example, one or more gene associated with the accumulation of cell wall components may be knocked out or have its expression otherwise silenced. A preferred example of a gene associated with accumulation of cell wall components, expression of which may beneficially be permanently reduced or eliminated in the yeast cells of the invention, is CHS3, which regulates chitin biosynthesis.

### “Digestive Tract”

The digestive tract, for the purposes of the present disclosure, may be taken as comprising a number of compartments, including the oral cavity, oesophagus, stomach, small intestine (which may be further divided into the duodenum, jejunum and ileum), large intestine (which may be further divided into the cecum, appendix and colon), and rectum. The digestive tract is sometimes divided into the upper digestive tract (comprising the oral cavity, oesophagus and stomach) and the lower digestive tract (comprising the small intestine, large intestine and rectum).

Administration to the digestive tract may preferably be by oral administration, though other alternative routes are also to be encompassed, as appropriate.

Promoters may be selected which respond to conditions found in any of the compartments of the digestive tract. Similarly, yeast cells of the invention may be targeted to lyse in any of these compartments. However, there are a number of considerations that make certain compartments of the digestive tract more favoured sites for targeted cell lysis than others. For example, the conditions found in the stomach may significantly degrade protein agents if lysis occurs in this compartment. Accordingly, while it may be preferred to utilise promoters that will respond to conditions in the stomach to initiate the changes in gene expression that will eventually lead to yeast cell lysis, it may be preferred that this lysis occurs in the a “downstream” compartment, rather than in the stomach itself.

Furthermore, it may be preferred that promoters are selected which respond to conditions found in those compartments of the digestive tract associated with protein uptake. Thus the duodenum, jejunum, ileum and cecum may all represent preferred digestive tract compartments to which yeast cell lysis may be targeted by the use of suitable promoters.

Of the digestive tract compartments referred to above, the duodenum is the compartment through which most orally delivered drugs are absorbed. Accordingly, it may be most preferred that yeast cells in accordance with the present invention are targeted to lyse here. The means by which such targeting to this compartment, or the other compartments contemplated, may be achieved are described further elsewhere in the specification.

### “Promoters”

Promoters regulate gene expression. They may increase or decrease expression of a gene in response to different stimuli. The inventors have identified a number of genes with promoters that naturally respond to conditions found in the digestive tract. These include promoters that down-regulate gene expression in response to such conditions (i.e. the level of gene expression is decreased in response to exposure to the relevant conditions) and promoters that up-regulate gene expression in response to such conditions (i.e. expression is increased in response to exposure to the relevant conditions).

Yeast cells of the invention may include multiple copies of one particular chosen promoter, each of which is operatively linked to a copy of a gene associated with yeast cell wall integrity. Alternatively, a suitable arrangement may include a number of different genes associated with yeast cell integrity, each operatively to a copy of different chosen promoters responsive to conditions characteristic of the digestive tract.

### “Operatively Linked”

For the purposes of the present disclosure, a gene and promoter may be considered to be operatively linked if the expression of the gene is influenced by the activity of the promoter. A promoter may either increase or decrease expression of a gene to which it is operatively linked.

### “Conditions Characteristic of the Digestive Tract”

Conditions characteristic of the digestive tract may include physical and/or environmental conditions found in the digestive tract, to which yeast cells in the digestive tract may be exposed. They may also include specific physical and/or environmental conditions to which yeast cells in one or more specific compartment(s) in the digestive tract are exposed.

Suitable parameters that may be considered when determining conditions characteristic of the digestive tract, or one or more specific compartments thereof, may be selected from the group consisting of: pH; temperature; salinity; shear forces; digestive enzymes; nutrients and surface active agents. Depending on the parameter in question, suitable assessments may be whether or not a given parameter is present (e.g. are surface active agents present?), or what the value is of a selected parameter (e.g. what is the pH?).

Among the digestive enzymes contributing to conditions characteristic of the digestive tract are: pepsin; gastric lipase pancreatic lipase; colipase; trypsin; and chymotrypsin. All of these enzymes may be found within one or more compartments of the digestive tract. Thus the yeast cells and artificial nucleic acid constructs of the invention may utilise promoters that alter gene expression in response to one or more of these digestive enzymes.

Surface active agents characteristic of the digestive tract include bile salts. Examples of such bile salts include sodium taurocholate and/or sodium glycocholate. The yeast cells and artificial nucleic acid constructs of the invention may utilise promoters that alter gene expression in response to one or more of these surface active agent.

Conditions characteristic of the digestive tract may preferably be characteristic of one or more of the compartments within the digestive tract. Thus suitable promoters may be independently selected from the group consisting of: promoters responsive to conditions characteristic of the oral cavity; promoters responsive to conditions characteristic of the oesophagus; promoters responsive to conditions characteristic of the stomach; promoters responsive to conditions characteristic of the small intestine, including one or more of the duodenum, jejunum and ileum; promoters responsive to conditions characteristic of the large intestine, including one or more of the cecum, appendix or colon; and promoters responsive to conditions characteristic of the rectum.

By way of example, conditions characteristic of the stomach may include one or more condition selected from the group consisting of: very low pH (around 2.5); high temperature (approximately 37° C.); the presence of digestive enzymes selected from gastric lipase and/or pepsin; and the presence of nutrients (such as fats, carbohydrates, minerals and/or vitamins). It may be preferred that a promoter responsive to conditions in the stomach varies gene expression in response to two, three or four of these conditions.

Conditions characteristic of the duodenum may include one or more selected from the group consisting of: slightly reduced pH (around 6.5); high temperature (approximately 37° C.); the presence of the digestive enzymes selected from trypsin, chymotrypsin, colipase and/or pancreatic lipase; the presence of calcium carbonate and the presence of bile salts. It may be preferred that a promoter responsive to conditions in the duodenum varies gene expression in response to two, three, four or five of these conditions.

Considerations for Preferred Targeting of Yeast Cell Lysis within the Digestive Tract

The very low pH found in the stomach means that acid-labile therapeutic agents exposed to these conditions are frequently degraded without having the opportunity to exert their desired activity. In contrast to the stomach, the contents of the duodenum, and the other intestinal compartments, are less subject to acid digestion. Furthermore, the intestines are well adapted to the uptake of compounds from within their lumens, and are provided with an extensive immune network (often referred to as the gut-associated lymphoid tissue, or GALT) able to generate both local and systemic immune responses. Accordingly the intestines represent a particularly preferred compartment of the digestive tract in which yeast cells in accordance with the invention may be induced to lyse.

It may be preferred that the selected promoters and genes are able to induce lysis in the small intestine, and more particularly the duodenum, since therapeutic agents released on lysis of yeast cells in this compartment (at the beginning of the intestines) will have maximum exposure to the intestines on their transit through the digestive tract, and thus have the greatest opportunity to exert their therapeutic effect.

It will be appreciated that, since orally administered yeast cells will pass through the various compartments of the digestive tract sequentially (from the mouth to the point at which cell lysis occurs) then promoters intended to induce yeast cell lysis in a selected compartment may be chosen on the basis of their response to the “cumulative” conditions to which they are exposed up until the point of lysis.

For example, in the case of yeast cells intended to undergo targeted lysis in the small intestine, it may be preferred to utilise a promoter that responds to conditions characteristic of the stomach and also responds (in the same way) to conditions characteristic of the small intestine. In particular, it may be preferred to utilise a promoter responsive to conditions characteristic of both the stomach and duodenum.

Promoters responsive to conditions found in the stomach are able to begin the biological processes leading to cell lysis (for instance down-regulation of yeast cell wall components, or up-regulation of cell wall-degrading agents) prior to the arrival of the yeast cells in the duodenum, thus providing a valuable “head start” to the subsequent rupture of the cells and release of the therapeutic agents. If the selected promoter is also responsive to conditions found in the duodenum, then the expression of genes associated with cell wall integrity, can continue in this compartment of the digestive tract. This can accelerate the process of cell lysis.

In a particularly preferred embodiment, it may be preferred to utilise at least one promoter that is responsive to conditions characteristic of the stomach, and that also exhibits that same response to conditions characteristic of the duodenum. In one embodiment such a promoter may up-regulate gene expression in response to conditions characteristic of the stomach, and also up-regulate expression in response to conditions characteristic of the duodenum. In a further embodiment such a promoter may down-regulate gene expression in response to conditions characteristic of the stomach, and also down-regulate expression in response to conditions characteristic of the duodenum.

The inventors have identified a number of genes with such promoters and these are set out in Tables 1 and 2. Table 1 sets out genes with promoters that up-regulate gene expression in response to conditions characteristic of the stomach and conditions characteristic of the duodenum. Table 2 sets out genes with promoters that down-regulate gene expression in response to conditions characteristic of the stomach and conditions characteristic of the duodenum. Promoters of any of the genes set out in Tables 1 and 2 may be used in the yeast cells or nucleic acids of the invention. In the case of promoters from the genes set out in Table 1 they may be operatively linked to genes that encode products associated with the degradation of cell wall components. In the case of promoters from the genes set out in Table 2, they may be operatively linked to genes that encode products associated with biogenesis of cell wall components.

TABLE 1
Genes whose transcript abundance is increased upon exposure to
gastric and duodenal conditions

| | | Fold | Fold | |
|---|---|---|---|---|
| ORF | Gene | (Gastric) | (Duodenal) | Description |
| YAL039C | CYC3 | 6.22 | 3.12 | Cytochrome c heme lyase |
| | | | | (holocytochrome c synthase) |
| YBL049W | MOH1 | 2.18 | 7.71 | Has homology to kinase Snf7p; |
| | | | | essential for viability in stationary |
| | | | | phase |
| YCL035C | GRX1 | 2.52 | 2.51 | Hydroperoxide and superoxide-radical |
| | | | | responsive heat-stable glutathione- |
| | | | | dependent disulfide oxidoreductase |
| YDL024C | DIA3 | 2.11 | 1.52 | Involved in invasive and |
| | | | | pseudohyphal growth |
| YDL079C | MRK1 | 3.55 | 3.71 | Glycogen synthase kinase 3 (GSK-3) |
| | | | | homolog |
| YDR070C | FMP16 | 5.79 | 25.33 | The authentic, non-tagged protein was |
| | | | | localized to the mitochondria |
| YER054C | GIP2 | 3.52 | 2.92 | Putative regulatory subunit of the |
| | | | | protein phosphatase Glc7p, involved |
| | | | | in glycogen metabolism |
| YFL007W | BLM10 | 2.27 | 1.31 | Proteosome activator subunit; required |
| | | | | for resistance to bleomycin |
| YFL009W | CDC4 | 1.38 | 1.69 | F-box protein required for G1/S and |
| | | | | G2/M transition |
| YFL030W | AGX1 | 4.63 | 22.13 | Alanine, glyoxylate aminotransferase, |
| | | | | catalyzes the synthesis of glycine from |
| | | | | glyoxylate |
| YFR033C | QCR6 | 2.75 | 4.16 | Subunit 6 of the ubiquinol |
| | | | | cytochrome-c reductase complex |
| YGL062W | PYC1 | 4.43 | 5.07 | Pyruvate carboxylase isoform, |
| | | | | cytoplasmic enzyme that converts |
| | | | | pyruvate to oxaloacetate |
| YGR174C | CBP4 | 2.89 | 2.04 | Mitochondrial protein required for |
| | | | | assembly of ubiquinol cytochrome-c |
| | | | | reductase complex |
| YGR239C | PEX21 | 3.72 | 3.50 | Part of a two-member peroxin family |
| | | | | (Pex18p and Pex21p) specifically |
| | | | | required for peroxisomal targeting of |
| | | | | the Pex7p peroxisomal signal |
| | | | | recognition factor and PTS2-type |
| | | | | peroxisomal matrix proteins |
| YGR248W | SOL4 | 12.49 | 3.74 | 6-phosphogluconolactonase with |
| | | | | similarity to Sol3p |
| YGR256W | GND2 | 19.19 | 5.92 | 6-phosphogluconate dehydrogenase |
| | | | | (decarboxylating) |
| YHR008C | SOD2 | 2.06 | 2.61 | Manganese-containing superoxide |
| | | | | dismutase; protects cells against |
| | | | | oxygen toxicity |
| YIL160C | POT1 | 1.86 | 54.05 | 3-ketoacyl-CoA thiolase with broad |
| | | | | chain length specificity |
| YIR038C | GTT1 | 3.56 | 2.85 | ER associated glutathione S- |
| | | | | transferase capable of |
| | | | | homodimerization |
| YJL161W | FMP33 | 2.96 | 2.61 | The authentic, non-tagged protein was |
| | | | | localized to the mitochondria |
| YJR104C | SOD1 | 3.87 | 1.83 | Cu, Zn superoxide dismutase; some |
| | | | | mutations are analogous to those that |
| | | | | cause ALS (amyotrophic lateral |
| | | | | sclerosis) in humans |
| YKL026C | GPX1 | 2.58 | 6.39 | Phospholipid hydroperoxide |
| | | | | glutathione peroxidase induced by |
| | | | | glucose starvation |
| YKR049C | FMP46 | 5.34 | 5.59 | Mitochondrial putative redox protein |
| | | | | containing a thioredoxin fold |
| YKR097W | PCK1 | 20.97 | 25.54 | Phosphoenolpyruvate carboxykinase, |
| | | | | key enzyme in gluconeogenesis |
| YLR107W | REX3 | 2.08 | 2.69 | RNA exonuclease; required for |
| | | | | maturation of the RNA component of |
| | | | | RNase MRP |
| YLR142W | PUT1 | 5.74 | 3.34 | Proline oxidase, nuclear-encoded |
| | | | | mitochondrial protein involved in |
| | | | | utilization of proline |
| YLR327C | TMA10 | 18.89 | 5.89 | Protein of unknown function that |
| | | | | associates with ribosomes |
| YLR375W | STP3 | 1.86 | 1.35 | Possibly involved in pre-tRNA |
| | | | | splicing and in uptake of branched- |
| | | | | chain amino acids |
| YLR377C | FBP1 | 2.52 | 58.57 | Fructose-1,6-bisphosphatase, key |
| | | | | regulatory enzyme in the |
| | | | | gluconeogenesis pathway |
| YML054C | CYB2 | 1.69 | 7.73 | Cytochrome b2 (L-lactate |
| | | | | cytochrome-c oxidoreductase) |
| YMR009W | ADI1 | 2.57 | 2.74 | Acireductone dioxygenease involved |
| | | | | in the methionine salvage pathway |
| YMR107W | SPG4 | 4.92 | 320.95 | Protein required for survival at high |
| | | | | temperature during stationary phase |
| YMR251W-A | HOR7 | 3.38 | 1.66 | Overexpression suppresses Ca2+ |
| | | | | sensitivity of mutants lacking inositol |
| | | | | phosphorylceramide |
| | | | | mannosyltransferases |
| YNL015W | PBI2 | 9.13 | 4.48 | Cytosolic inhibitor of vacuolar |
| | | | | proteinase B, required for efficient |
| | | | | vacuole inheritance |
| YNL063W | MTQ1 | 1.56 | 1.39 | S-adenosylmethionine-dependent |
| | | | | Methyltransferase; methylates |
| | | | | translational release factor Mrf1p |
| YOL052C-A | DDR2 | 39.98 | 14.76 | Multistress response protein, |
| | | | | expression is activated by a variety of |
| | | | | xenobiotic agents and environmental |
| | | | | or physiological stresses |
| YOL117W | RRI2 | 1.49 | 3.19 | Subunit of the COP9 signalosome |
| | | | | (CSN) complex that cleaves the |
| | | | | ubiquitin-like protein Nedd8 from |
| | | | | SCF ubiquitin ligases; plays a role in |
| | | | | the mating pheromone response |
| YOR173W | DCS2 | 7.83 | 2.99 | Non-essential protein containing a |
| | | | | HIT (histidine triad) motif |
| YOR328W | PDR10 | 3.69 | 20.86 | ABC (ATP-binding cassette) |
| | | | | membrane pump involved in the |
| | | | | pleiotropic drug resistance network |
| YOR348C | PUT4 | 6.64 | 16.66 | Proline permease, required for high- |
| | | | | affinity transport of proline |
| YPL024W | RMI1 | 4.04 | 2.36 | Involved in response to DNA damage |
| YPL240C | HSP82 | 8.85 | 1.41 | Cytoplasmic chaperone (Hsp90 |
| | | | | family) required for pheromone |
| | | | | signaling and negative regulation of |
| | | | | Hsf1p |
| YPR065W | ROX1 | 1.71 | 4.18 | Heme-dependent repressor of hypoxic |
| | | | | genes |

TABLE 2
Genes whose transcript abundance is decreased upon exposure to
gastric and duodenal conditions

| | | Fold | Fold | |
|---|---|---|---|---|
| ORF | Gene | (Gastric) | (Duodenal) | Description |
| YBR048W | RPS11B | −2.28 | −5.92 | Protein component of the small (40S) |
| | | | | ribosomal subunit |
| YCL005W-A | VMA9 | −1.57 | −1.33 | Vacuolar H+ ATPase |
| YCL064C | CHA1 | −2.57 | −12.37 | Catabolic L-serine (L-threonine) |
| | | | | deaminase |
| YER011W | TIRI | −13.65 | −9.57 | Cell wall mannoprotein of the |
| | | | | Srp1p/Tip1p family of serine-alanine- |
| | | | | rich proteins |
| YER131W | RPS26B | −2.08 | −8.90 | Protein component of the small (40S) |
| | | | | ribosomal subunit |
| YGL089C | MFα2 | −14.53 | −5.54 | Mating pheromone alpha-factor |
| YGR208W | SER2 | −2.19 | −3.27 | Phosphoserine phosphatase |
| YJR047C | ANB1 | −49.79 | −3.73 | Translation initiation factor eIF-5A |
| YMR091C | NPL6 | −1.62 | −1.61 | Component of the RSC chromatin |
| | | | | remodeling complex |
| YMR123W | PKR1 | −2.01 | −1.36 | Protein of unknown function |
| YNL087W | TCB2 | −1.69 | −1.96 | Bud-specific protein with a potential |
| | | | | role in membrane trafficking |
| YPL208W | RKM1 | −2.34 | −2.02 | SET-domain lysine-N- |
| | | | | methyltransferase |

While the preceding paragraphs describe generally preferred embodiments of the invention, there may be circumstances in which it is wished to use promoters that respond differently to conditions found in one digestive tract compartment (such as the stomach) than they do to conditions found in another digestive tract compartment (such as the duodenum). A single such promoter may be used to regulate expression of a gene associated with yeast cell wall integrity in only one digestive tract compartment. This may optionally be combined with one (or more) promoter(s) capable of regulating expression of a gene associated with cell wall integrity in the same compartment, or in different compartment(s).

### Examples of Preferred Promoters Responsive to Conditions Characteristic of the Digestive Tract

The inventors have found that only a very small number of genes alter their expression levels in response to conditions characteristic of the stomach. Out of the more than 6,000 genes found in yeast, the inventors have found that only 43 decrease and 115 increase their transcript abundance, respectively in response to these conditions. The promoters of any of these genes may potentially be of use in the yeast cells of the invention.

Of the genes found to have promoters responsive to conditions characteristic of the stomach, the inventors have identified 30 genes that demonstrate preferred levels of down-regulation (more than 2 fold) in response to stomach conditions, and 92 genes that demonstrate preferred levels of increased transcription (more than two fold difference) when yeast are exposed to such conditions. Details of these 122 genes are set out in Table 3. In Table 3 the genes are identified by means of the position of their open reading frame, and details are provided of the changes in expression observed. Genes that undergo up-regulation of transcription are shown in the “Up” column (as well as the fold-increase in transcription), while genes that are down-regulated are shown in the “Down” column (as well as their fold-decrease in transcription levels).

Any of the genes set out in the “Down” column of Table 3 may provide promoters suitable for use in the yeast cells or nucleic acids of the invention. Suitable promoters selected from this group may be operatively linked to one or more genes associated with the accumulation of yeast cell wall components. Such genes may include SRB1 or PKC1, both of which are considered in more detail elsewhere in the specification.

TABLE 3
Genes responsive to conditions characteristic of the stomach

| | |
|---|---|
| UP 92 | DOWN 30 |

| UP | ORF | DOWN | ORF |
|---|---|---|---|
| 846.3074581 | YFL014W | −2.0123 | YMR123W |
| 77.19194828 | YOL151W | −2.07795 | YER131W |
| 49.43240015 | YDR533C | −2.19459 | YGR208W |
| 39.97794367 | YOL052C-A | −2.2828 | YBR048W |
| 20.9716449 | YKR097W | −2.33761 | YPL208W |
| 19.49327072 | YGR043C | −2.54376 | YPL075W |
| 19.18602458 | YGR256W | −2.56615 | YCL064C |
| 18.88993375 | YLR327C | −2.5708 | YOL092W |
| 15.98532569 | YMR169C | −2.77384 | YLR214W |
| 15.92112432 | YNL134C | −2.78575 | YLR450W |
| 13.53084044 | YDR453C | −2.79742 | YIR033W |
| 12.4972339 | YGR248W | −2.81194 | YMR321C |
| 12.25888186 | YDR034W-B | −2.99914 | YLR228C |
| 11.83826262 | YAL005C | −3.12331 | YPR158W |
| 9.972910717 | YEL059C-A | −3.19486 | YIL114C |
| 9.568716622 | YCL026C-B | −3.24143 | YDR056C |
| 9.13461433 | YNL015W | −3.36282 | YJL047C-A |
| 9.100036761 | YHR033W | −3.68899 | YGL255W |
| 8.846438493 | YPL240C | −3.89561 | YGR108W |
| 7.832518776 | YOR173W | −4.57649 | YPR124W |
| 7.240316316 | YBR244W | −5.44467 | YIL119C |
| 7.187138594 | YFR053C | −6.19885 | YBR066C |
| 7.068141765 | YJR096W | −10.8508 | YJR150C |
| 6.873698099 | YMR090W | −11.3352 | YBR067C |
| 6.742344365 | YDL244W | −13.6453 | YER011W |
| 6.636711768 | YOR348C | −14.1215 | YHL028W |
| 6.225835458 | YAL039C | −14.5389 | YGL089C |
| 6.183014778 | YER103W | −17.163 | YJR004C |
| 6.144708541 | YMR316W | −28.3954 | YMR319C |
| 6.118441921 | YMR173W | −38.4641 | YDR044W |
| 6.087271751 | YHR138C | −49.7888 | YJR047C |
| 5.792976647 | YDR070C | | |
| 5.736024404 | YLR142W | | |
| 5.623007069 | YLR259C | | |
| 5.508826756 | YJL088W | | |
| 5.382600609 | YLR109W | | |
| 5.347989991 | YKR049C | | |
| 5.175037602 | YIL014C-A | | |
| 4.923784758 | YMR107W | | |
| 4.759486603 | YMR189W | | |
| 4.691008484 | YDR214W | | |
| 4.648760305 | YEL066W | | |
| 4.648406574 | YPL111W | | |
| 4.62673528 | YFL030W | | |
| 4.431610954 | YGL062W | | |
| 4.39918813 | YGR209C | | |
| 4.345712512 | YOR289W | | |
| 4.197358204 | YJL052W | | |
| 4.144017878 | YNR034W-A | | |
| 4.040983419 | YPL024W | | |
| 4.023579803 | YDR342C | | |
| 3.865512625 | YJR104C | | |
| 3.786061905 | YIL117C | | |
| 3.735122446 | YDR270W | | |
| 3.720511672 | YGR239C | | |
| 3.708004419 | YHR054C | | |
| 3.695250308 | YOR328W | | |
| 3.630486311 | YBR285W | | |
| 3.556387552 | YIR038C | | |
| 3.551625667 | YDL079C | | |
| 3.516199671 | YER054C | | |
| 3.405740569 | YOR285W | | |
| 3.376873581 | YMR251W-A | | |
| 3.273835104 | YCL036W | | |
| 3.265004528 | YJL052W | | |
| 3.135850421 | YFR011C | | |
| 3.114258607 | YLL009C | | |
| 3.098143751 | YKR091W | | |
| 3.057086395 | YBL029C-A | | |
| 3.028068249 | YML131W | | |
| 2.989342629 | YCR076C | | |
| 2.986420342 | YGL064C | | |
| 2.961642592 | YJL161W | | |
| 2.899332925 | YGR174C | | |
| 2.79882596 | YGR021W | | |
| 2.7491496 | YFR033C | | |
| 2.672284645 | YHR030C | | |
| 2.581087237 | YKL026C | | |
| 2.573573855 | YMR009W | | |
| 2.520568521 | YCL035C | | |
| 2.520382041 | YLR377C | | |
| 2.383925796 | YGL091C | | |
| 2.298298033 | YDL144C | | |
| 2.268884514 | YFL007W | | |
| 2.268230818 | YBR267W | | |
| 2.218706089 | YAL058W | | |
| 2.185631529 | YBR137W | | |
| 2.1833919 | YBL049W | | |
| 2.132621926 | YOR201C | | |
| 2.114344289 | YDL024C | | |
| 2.07527051 | YLR107W | | |
| 2.05977966 | YHR008C | | |
| 2.058137833 | YCR046C | | |

It is interesting to note that, of the genes (and hence gene promoters) identified in the “Up” column, eleven have no known molecular function. Accordingly, there can be no question of there having been any previous indications that these promoters may be used to regulate gene expression in response to conditions characteristic of the digestive tract (and particularly of the stomach). These genes are: YDR056C, YDR063W, YJL047C-A, DAN1, YLR065C, PKR1, YMR321C, TCB2, YOL092W, YPRO89W, and YPR158W.

Any of the genes set out in the “Up” column of Table 3 may provide promoters suitable for use in the yeast cells or nucleic acids of the invention. Suitable promoters of one or more of the genes selected from this group may be operatively linked to one or more genes associated with degradation of yeast cell wall components. Genes that may be operatively linked to such promoters include those encoding cell wall component-degrading enzymes such as endochitinases (encoded by genes such as CTS1) or exo, 1-3β-glucanase (encoded by genes such as EXG1).

The inventors have also identified a small number of genes whose transcript abundance alters in response to conditions characteristic of the duodenum. Of the more than 6,000 genes expressed by yeast, the inventors have found that 460 decrease, and 459 increase their expression levels on exposure to conditions characteristic of the duodenum. The promoters of any of these genes may potentially be of use in the yeast cells of the invention.

Of the genes found to have promoters responsive to conditions characteristic of the duodenum, the inventors have identified 386 genes that demonstrate preferred levels of down-regulation in response to stomach conditions, and 391 genes that demonstrate preferred levels of increased transcription when yeast are exposed to such conditions. Details of these 777 genes are set out in Table 4. In Table 4 the genes are identified by means of the position of their open reading frame, and details are provided of the changes in expression observed. Genes that undergo up-regulation of transcription are shown in the “Up” column (as well as the fold-increase in transcription), while genes that are down-regulated are shown in the “Down” column (as well as their fold-decrease in transcription).

Genes identified in the “Down” column of Table 4 will have promoters suitable to be operatively linked to one or more genes associated with the accumulation of yeast cell wall components. As before, such genes may include SRB1, or PKC1.

TABLE 4
Genes responsive to conditions characteristic of the small intestine

| UP 391 | | DOWN 386 | |
|---|---|---|---|
| UP | ORF | DOWN | ORF |
| 813.5264099 | YNL117W | −2.000345569 | YDR089W |
| 320.9539758 | YMR107W | −2.005157815 | YMR024W |
| 306.6663448 | YGR236C | −2.005967721 | YPL028W |
| 267.2823519 | YIL057C | −2.01620826 | YPL208W |
| 219.1548034 | YMR175W | −2.019322497 | YKL130C |
| 202.9162822 | YLR307C-A | −2.02120433 | YDL051W |
| 170.2056018 | YPL223C | −2.030681185 | YPL217C |
| 125.8114835 | YJR095W | −2.033984133 | YGR200C |
| 117.8686248 | YER024W | −2.048376843 | YDL040C |
| 113.9247964 | YKL217W | −2.064548622 | YIL091C |
| 95.42924489 | YER065C | −2.065383732 | YDR365C |
| 93.39108987 | YAR035W | −2.067814164 | YHR062C |
| 93.20905875 | YCR010C | −2.069493796 | YPL211W |
| 90.44008531 | YMR303C | −2.078699289 | YDR062W |
| 79.61201726 | YDL085W | −2.080729882 | YJR014W |
| 79.27122716 | YHR096C | −2.091139957 | YML092C |
| 71.07435538 | YAL054C | −2.099536772 | YPR004C |
| 62.82545649 | YBR296C | −2.101177989 | YGL099W |
| 60.2294407 | YLR174W | −2.115905894 | YPL037C |
| 59.9658359 | YHR139C | −2.118120355 | YLR017W |
| 58.5682477 | YLR377C | −2.122949279 | YPL111W |
| 54.04841809 | YIL160C | −2.124069451 | YDR339C |
| 51.92459851 | YMR118C | −2.129748918 | YLR051C |
| 47.76600474 | YNR002C | −2.163666277 | YGR245C |
| 46.36057995 | YDR256C | −2.166861896 | YJR001W |
| 41.53241728 | YPR002W | −2.17980415 | YDR398W |
| 40.20113773 | YOR100C | −2.184884651 | YJL122W |
| 37.57881388 | YCR005C | −2.185630438 | YER023W |
| 32.97955322 | YBL075C | −2.187052363 | YDR487C |
| 30.97126968 | YJL153C | −2.198837508 | YNL153C |
| 27.87508901 | YPL054W | −2.201633659 | YDR395W |
| 27.13999457 | YMR206W | −2.207028214 | YMR128W |
| 26.28992662 | YAL062W | −2.209420174 | YIL148W |
| 25.53944826 | YKR097W | −2.21133238 | YIR008C |
| 25.32727059 | YDR070C | −2.225397632 | YFR051C |
| 24.73330572 | YNL014W | −2.228496516 | YDR342C |
| 24.51299794 | YBL015W | −2.242545526 | YBR246W |
| 24.33548356 | YDR034W-B | −2.243793046 | YLR134W |
| 22.92775753 | YOL126C | −2.25076451 | YDL167C |
| 22.12572157 | YFL030W | −2.275855637 | YIL070C |
| 21.73733827 | YML042W | −2.27886826 | YLL025W |
| 21.21662086 | YGR067C | −2.291249965 | YNR054C |
| 20.86230343 | YOR328W | −2.291385149 | YNL151C |
| 20.28700547 | YNL195C | −2.297076847 | YLR259C |
| 19.21081634 | YPL201C | −2.298063255 | YDR245W |
| 16.65704831 | YOR348C | −2.302798541 | YJL008C |
| 15.96790447 | YJL133C-A | −2.304315331 | YPL129W |
| 15.92713581 | YKR009C | −2.315034466 | YIL134W |
| 15.7311733 | YKL107W | −2.347861192 | YDL061C |
| 15.28550256 | YDR171W | −2.353989783 | YDR165W |
| 14.76146837 | YOL052C-A | −2.367960108 | YIR021W |
| 14.42383635 | YPR001W | −2.379790651 | YKL172W |
| 14.30148818 | YGR121C | −2.379982608 | YPL245W |
| 13.77762719 | YOL084W | −2.387486444 | YPR183W |
| 13.46443214 | YLR267W | −2.398900227 | YGL232W |
| 13.3983511 | YKL187C | −2.406085791 | YMR230W |
| 13.22823213 | YLR149C | −2.42184411 | YDR119W |
| 13.13194076 | YMR280C | −2.425570504 | YDR429C |
| 12.86707778 | YNL194C | −2.429083473 | YOR046C |
| 12.58509055 | YOR031W | −2.429167028 | YDL191W |
| 12.41618523 | YNL277W | −2.431574898 | YDR083W |
| 12.09962924 | YBR117C | −2.435146412 | YER019W |
| 11.89279091 | YGR142W | −2.44000858 | YOR168W |
| 11.77617513 | YDL214C | −2.440360363 | YJR063W |
| 11.73939779 | YPL147W | −2.44085593 | YAL025C |
| 11.70789486 | YBR230C | −2.464305088 | YBR096W |
| 11.538807 | YMR256C | −2.473507803 | YFR009W |
| 11.52377951 | YIL136W | −2.478996503 | YNL070W |
| 10.77250897 | YJL089W | −2.501919859 | YPL106C |
| 10.57624081 | YFR017C | −2.504756684 | YPL127C |
| 10.54611465 | YLR312C | −2.508767562 | YLR106C |
| 10.31874482 | YDL204W | −2.516562651 | YPL144W |
| 10.28530344 | YBR085C-A | −2.527526469 | YMR076C |
| 10.22979077 | YIL155C | −2.547882503 | YPL252C |
| 10.08433833 | YPR151C | −2.571247663 | YBR073W |
| 9.981106914 | YPL222W | −2.574627369 | YDR238C |
| 9.719797602 | YKR039W | −2.581008471 | YBR283C |
| 9.616640917 | YDR384C | −2.586855244 | YGL195W |
| 9.044776496 | YBR050C | −2.586984179 | YKL014C |
| 8.884374478 | YLR053C | −2.611769628 | YAL038W |
| 8.423235679 | YHR140W | −2.623848151 | YGL189C |
| 8.238227409 | YLR038C | −2.63177628 | YBR243C |
| 8.13294275 | YMR323W | −2.637361327 | YOR272W |
| 8.026773026 | YKL171W | −2.64562929 | YDR309C |
| 8.018461504 | YHR138C | −2.647737489 | YJL148W |
| 7.936549648 | YDR178W | −2.650757334 | YML074C |
| 7.935273211 | YOL083W | −2.679746286 | YFL034C-A |
| 7.830461716 | YGR110W | −2.679785009 | YGL030W |
| 7.767830487 | YMR085W | −2.707197423 | YMR267W |
| 7.746897263 | YNR001C | −2.708419864 | YBR175W |
| 7.732445971 | YER150W | −2.717260601 | YJL050W |
| 7.730617427 | YML054C | −2.719455786 | YDR234W |
| 7.707176638 | YBL049W | −2.720304904 | YLL034C |
| 7.66926457 | YPL156C | −2.720612892 | YNL178W |
| 7.639893646 | YOR186W | −2.72759017 | YDR060W |
| 7.56405294 | YJL144W | −2.738223021 | YLR129W |
| 7.522972335 | YNL093W | −2.747521338 | YOR167C |
| 7.506923445 | YDL215C | −2.750595832 | YFR032C-A |
| 7.499009834 | YJR008W | −2.781308261 | YGR279C |
| 7.429816892 | YAL061W | −2.800460129 | YHR025W |
| 7.312353679 | YGL205W | −2.806056774 | YGR085C |
| 7.278361099 | YKL141W | −2.819248307 | YKL080W |
| 7.266140303 | YNR034W-A | −2.835972125 | YIL094C |
| 7.215436081 | YJL045W | −2.84868906 | YGL026C |
| 7.09901902 | YJL048C | −2.861285318 | YOR224C |
| 7.030914935 | YKL198C | −2.863375295 | YHL001W |
| 6.833722443 | YLR414C | −2.877010798 | YLR222C |
| 6.779272299 | YGR183C | −2.879199676 | YDL112W |
| 6.748742456 | YGL208W | −2.88429396 | YCL059C |
| 6.699833108 | YDL222C | −2.888331035 | YOR335C |
| 6.682806784 | YDR406W | −2.896690501 | YHR021C |
| 6.567021639 | YJR115W | −2.896995012 | YBL092W |
| 6.528080434 | YLL061W | −2.917046948 | YCL036W |
| 6.5207448 | YHR097C | −2.925953626 | YMR230W |
| 6.399678993 | YKL026C | −2.927378861 | YKR026C |
| 6.39776847 | YLR346C | −2.932628218 | YGR027C |
| 6.390044833 | YDR525W-A | −2.936193016 | YBR267W |
| 6.370732218 | YCR021C | −2.944906465 | YOL123W |
| 6.248878967 | YBR067C | −2.949151078 | YNL061W |
| 6.183960639 | YLR164W | −2.97940426 | YDR410C |
| 6.157264919 | YML128C | −2.98137996 | YDL103C |
| 6.144523637 | YOR220W | −2.981984217 | YEL001C |
| 6.1140764 | YDL130W-A | −2.983727654 | YNL062C |
| 6.100488099 | YFR049W | −2.984768257 | YHR013C |
| 6.077135094 | YGR174W-A | −2.995109949 | YBL024W |
| 6.043663742 | YKL093W | −2.99730239 | YGL001C |
| 6.036468025 | YJL163C | −2.999511782 | YNL102W |
| 6.019069565 | YGR088W | −3.00408107 | YPR069C |
| 5.983633004 | YMR322C | −3.046654749 | YDR500C |
| 5.917253895 | YGR256W | −3.054371353 | YFL022C |
| 5.899460561 | YKR076W | −3.061653086 | YGL189C |
| 5.898978523 | YLR327C | −3.075259675 | YIL074C |
| 5.870684161 | YMR040W | −3.077859587 | YLR195C |
| 5.823977269 | YMR175W-A | −3.094147896 | YDR454C |
| 5.792251446 | YDL169C | −3.098433054 | YER090W |
| 5.754073688 | YDL149W | −3.118501149 | YMR321C |
| 5.730295934 | YIL172C | −3.128225971 | YDR450W |
| 5.683439523 | YDR529C | −3.131114581 | YBR041W |
| 5.674570824 | YEL012W | −3.13192713 | YHR038W |
| 5.655613167 | YHL032C | −3.135055786 | YMR202W |
| 5.637534682 | YNL202W | −3.135864634 | YKR057W |
| 5.588204148 | YKR049C | −3.137768979 | YHR197W |
| 5.449964214 | YOL081W | −3.147262516 | YOL097C |
| 5.44116889 | YMR034C | −3.153029962 | YDL075W |
| 5.403535803 | YPL271W | −3.168575612 | YLR042C |
| 5.321691902 | YCR007C | −3.18382424 | YCR072C |
| 5.320965746 | YDL199C | −3.186253337 | YPR060C |
| 5.303183832 | YIL146C | −3.19177603 | YGL171W |
| 5.146927413 | YNL036W | −3.202480246 | YLL012W |
| 5.142201234 | YML091C | −3.212054148 | YLR449W |
| 5.07791043 | YMR081C | −3.21276437 | YOL056W |
| 5.069820227 | YGL062W | −3.223005486 | YMR214W |
| 5.062034727 | YLR054C | −3.231537731 | YLR264W |
| 5.044051977 | YPL250C | −3.238213843 | YLR432W |
| 5.032352914 | YMR182W-A | −3.246097951 | YPR033C |
| 4.881049917 | YDR018C | −3.266165018 | YGR208W |
| 4.84538486 | YPL274W | −3.277400066 | YHR193C |
| 4.827313632 | YBR241C | −3.308112011 | YGL135W |
| 4.8256757 | YMR181C | −3.311274968 | YER049W |
| 4.824358336 | YMR136W | −3.322869952 | YBR244W |
| 4.81734805 | YFR029W | −3.350184658 | YHR141C |
| 4.805531651 | YPR020W | −3.356434595 | YPL093W |
| 4.80334278 | YGR230W | −3.385398707 | YML124C |
| 4.773348884 | YDR253C | −3.385863031 | YJR064W |
| 4.729869855 | YGR243W | −3.415530795 | YMR318C |
| 4.719548437 | YHL021C | −3.420735121 | YGR078C |
| 4.716570211 | YJL103C | −3.429585306 | YLR276C |
| 4.640101106 | YGL045W | −3.437935081 | YLR388W |
| 4.61430378 | YGR144W | −3.441320875 | YBR187W |
| 4.61363204 | YPL171C | −3.444983165 | YGL076C |
| 4.607569453 | YIL101C | −3.451033455 | YMR049C |
| 4.581334399 | YDR148C | −3.461749235 | YAL023C |
| 4.579996849 | YGR070W | −3.478797734 | YOR198C |
| 4.565339549 | YMR191W | −3.509463132 | YLR244C |
| 4.553178512 | YDR425W | −3.51258944 | YLR196W |
| 4.535820999 | YLR345W | −3.558031733 | YBL072C |
| 4.527206958 | YNL277W-A | −3.579352222 | YMR078C |
| 4.521280324 | YDR043C | −3.597363531 | YBR143C |
| 4.496048593 | YKL185W | −3.597655092 | YDL084W |
| 4.483039274 | YEL060C | −3.600895107 | YHR196W |
| 4.482210343 | YNL015W | −3.602861492 | YGL097W |
| 4.455310705 | YLR001C | −3.605268328 | YDR518W |
| 4.40088679 | YMR114C | −3.639890492 | YLR103C |
| 4.378376992 | YER033C | −3.640919068 | YDR226W |
| 4.371691144 | YHR051W | −3.65489745 | YNL067W |
| 4.367589806 | YHR160C | −3.66498343 | YOR340C |
| 4.333231297 | YIL087C | −3.689369475 | YNL220W |
| 4.298359961 | YKL086W | −3.692251235 | YOR206W |
| 4.285908094 | YBL029C-A | −3.697320908 | YBL069W |
| 4.205301917 | YDR322C-A | −3.734898514 | YLR388W |
| 4.180549487 | YPR065W | −3.734900375 | YJR047C |
| 4.165249874 | YBR033W | −3.74043145 | YML008C |
| 4.163532855 | YKL032C | −3.755859503 | YEL026W |
| 4.16250718 | YFR033C | −3.764406719 | YEL042W |
| 4.145389428 | YJL102W | −3.771415684 | YFR053C |
| 4.131351206 | YJR073C | −3.771804138 | YOR222W |
| 4.122137935 | YIL065C | −3.802803499 | YLR409C |
| 4.105410834 | YNL196C | −3.814810574 | YLR406C |
| 4.060139531 | YPL262W | −3.841396411 | YPR133W-A |
| 4.057931003 | YOR023C | −3.866445475 | YML026C |
| 4.029304217 | YBR089C-A | −3.868549751 | YML022W |
| 4.023402039 | YBR089C-A | −3.873526361 | YBR106W |
| 4.004837652 | YMR110C | −3.888058293 | YGL245W |
| 4.004645945 | YIL105C | −3.889728402 | YKL127W |
| 3.975779675 | YPR149W | −3.90623425 | YGR124W |
| 3.973897605 | YIL113W | −3.920997127 | YJL109C |
| 3.973217829 | YIL055C | −3.922231505 | YPL163C |
| 3.936233327 | YBR269C | −3.922559282 | YER156C |
| 3.930355709 | YMR271C | −3.928947501 | YHR019C |
| 3.914953348 | YAL034C | −3.932074 | YGL103W |
| 3.906519829 | YNL278W | −3.952065614 | YOR234C |
| 3.905659167 | YGR146C | −3.955169516 | YNR050C |
| 3.885282718 | YNL104C | −3.959718837 | YDR471W |
| 3.866056599 | YOR035C | −3.960248134 | YDR353W |
| 3.857019397 | YKL016C | −3.973600603 | YEL055C |
| 3.853650753 | YJL170C | −3.982906631 | YPL143W |
| 3.839993843 | YLR350W | −4.006245536 | YDR158W |
| 3.804614647 | YBR101C | −4.006846705 | YKL056C |
| 3.798622016 | YGR130C | −4.010387788 | YER117W |
| 3.795869919 | YMR041C | −4.020194974 | YKR094C |
| 3.774408922 | YFL021W | −4.028323247 | YKR080W |
| 3.762716524 | YLR228C | −4.035888967 | YOR293W |
| 3.758983128 | YDR319C | −4.052784301 | YDR144C |
| 3.758194319 | YGR043C | −4.066253778 | YMR194W |
| 3.755824144 | YPR098C | −4.091865527 | YBR121C |
| 3.742562325 | YGR248W | −4.094693036 | YAL044C |
| 3.737646465 | YKL146W | −4.098955202 | YHR203C |
| 3.72040961 | YEL073C | −4.135985732 | YGL234W |
| 3.710916423 | YDL079C | −4.182862366 | YDR321W |
| 3.709955322 | YDL200C | −4.186532015 | YLR179C |
| 3.708518289 | YGL191W | −4.190116223 | YBR092C |
| 3.64122817 | YLR136C | −4.209571628 | YDR447C |
| 3.588595872 | YIL046W | −4.228188464 | YJR069C |
| 3.58285718 | YDR461W | −4.236471436 | YBR084C-A |
| 3.565396195 | YDR244W | −4.250751921 | YCR031C |
| 3.549970285 | YMR016C | −4.258227021 | YDR400W |
| 3.501015228 | YGR239C | −4.289324345 | YNL175C |
| 3.476014209 | YKL188C | −4.295303486 | YHR163W |
| 3.447915474 | YPR006C | −4.303859535 | YLR167W |
| 3.434846745 | YNL192W | −4.309775548 | YJR094W-A |
| 3.43420642 | YHR124W | −4.321978078 | YML125C |
| 3.413505199 | YHR161C | −4.328632272 | YBL087C |
| 3.40696034 | YOR327C | −4.342361022 | YER056C-A |
| 3.401742668 | YNL200C | −4.348435678 | YGR285C |
| 3.370462136 | YOR228C | −4.353976176 | YMR217W |
| 3.348180092 | YDR329C | −4.360079151 | YAR002C-A |
| 3.343737695 | YOR020W-A | −4.363236392 | YIL052C |
| 3.335038973 | YLR142W | −4.366321704 | YAR075W |
| 3.324455737 | YOL100W | −4.397745316 | YDR087C |
| 3.292278849 | YPL055C | −4.469665514 | YAL007C |
| 3.290324396 | YHL020C | −4.476846027 | YDR037W |
| 3.275863448 | YBR140C | −4.502905729 | YGR103W |
| 3.275348446 | YML087C | −4.508826232 | YGL012W |
| 3.230242227 | YLR152C | −4.51101294 | YIL145C |
| 3.198465622 | YOL117W | −4.52125836 | YLR075W |
| 3.195422013 | YJL141C | −4.526045298 | YHR020W |
| 3.174510736 | YIL045W | −4.578706906 | YGL148W |
| 3.170812889 | YBR132C | −4.587341638 | YDL192W |
| 3.168472278 | YDR513W | −4.598518923 | YBR084W |
| 3.163663193 | YJL016W | −4.605722419 | YNL112W |
| 3.141856734 | YDL194W | −4.620537749 | YDR025W |
| 3.131253507 | YPR093C | −4.622388421 | YDR385W |
| 3.130907558 | YML076C | −4.658616697 | YGR061C |
| 3.118173207 | YAL039C | −4.718024026 | YGL123W |
| 3.107058322 | YOR382W | −4.723318435 | YFL045C |
| 3.091990921 | YKL038W | −4.752709034 | YNL302C |
| 3.091471573 | YNL305C | −4.78475741 | YGR177C |
| 3.090361075 | YDL110C | −4.787386848 | YOR369C |
| 3.085020019 | YDL174C | −4.792586197 | YEL047C |
| 3.052649271 | YER162C | −4.808692374 | YMR143W |
| 3.026627389 | YBR294W | −4.813525157 | YOR293W |
| 3.016945715 | YML081C-A | −4.826794875 | YJL145W |
| 3.0112805 | YKL085W | −4.84999727 | YLR185W |
| 2.990867001 | YOR173W | −4.950849935 | YGL202W |
| 2.985744904 | YNL157W | −4.962992844 | YOL121C |
| 2.982824316 | YDR231C | −4.977065002 | YLR340W |
| 2.982286522 | YPL078C | −4.978879956 | YBR191W |
| 2.969812199 | YDR359C | −5.02242695 | YOL086C |
| 2.967150778 | YBR047W | −5.031370511 | YJR070C |
| 2.965262201 | YNL092W | −5.038496962 | YAL012W |
| 2.95484564 | YNL077W | −5.1083256 | YPL160W |
| 2.946811133 | YDR169C | −5.108390498 | YLR197W |
| 2.937207249 | YMR204C | −5.119767554 | YBR031W |
| 2.928565961 | YOR285W | −5.150927126 | YJR123W |
| 2.920356509 | YER054C | −5.165183685 | YDR341C |
| 2.907685163 | YMR139W | −5.187640819 | YBR181C |
| 2.893498521 | YLR352W | −5.239337428 | YOL127W |
| 2.887148455 | YIL112W | −5.263321823 | YOR074C |
| 2.869380566 | YNL116W | −5.278338508 | YER074W |
| 2.86428348 | YDR530C | −5.280961145 | YIL133C |
| 2.86130337 | YPR061C | −5.308921238 | YLR343W |
| 2.854041145 | YIR038C | −5.318530027 | YKL128C |
| 2.82709816 | YBR016W | −5.321160295 | YOR108W |
| 2.826527273 | YHR136C | −5.329778108 | YOL040C |
| 2.814055011 | YDL176W | −5.429138576 | YOR306C |
| 2.807898772 | YJR036C | −5.449252993 | YHL033C |
| 2.806875727 | YDR262W | −5.457342585 | YML073C |
| 2.804803002 | YJR151C | −5.460930186 | YJL136C |
| 2.794717496 | YBR139W | −5.535663943 | YGL089C |
| 2.792001943 | YHR080C | −5.561723437 | YHR144C |
| 2.790943269 | YLR012C | −5.575880392 | YMR215W |
| 2.789749172 | YBR296C-A | −5.625573393 | YMR143W |
| 2.785713327 | YNL142W | −5.64280418 | YBR252W |
| 2.781849141 | YMR053C | −5.649399948 | YJL158C |
| 2.779595749 | YDR436W | −5.699955515 | YBR249C |
| 2.772796791 | YPL018W | −5.734197343 | YML026C |
| 2.770668865 | YOR084W | −5.753174183 | YHL033C |
| 2.753719108 | YNL097C | −5.803451957 | YDR064W |
| 2.739385395 | YMR009W | −5.813703254 | YEL040W |
| 2.736249526 | YHR006W | −5.861873514 | YFR031C-A |
| 2.734592481 | YOR036W | −5.875200975 | YML063W |
| 2.723139365 | YER079W | −5.914569577 | YHR089C |
| 2.695785646 | YLR107W | −5.917883797 | YBR048W |
| 2.692123156 | YJR107W | −5.950032184 | YGR148C |
| 2.671464867 | YGR237C | −5.973737361 | YGR094W |
| 2.662411855 | YOL147C | −5.974306145 | YHR010W |
| 2.649090237 | YGL248W | −5.981773565 | YHR174W |
| 2.644674808 | YER144C | −5.992730315 | YML024W |
| 2.639974108 | YLR256W | −6.015143662 | YPL079W |
| 2.624645482 | YBR219C | −6.098152129 | YNL301C |
| 2.621843285 | YCL057C-A | −6.120544094 | YDL055C |
| 2.614313366 | YHR008C | −6.136218949 | YDL130W |
| 2.611775156 | YJL161W | −6.154401764 | YOR310C |
| 2.562109464 | YLR080W | −6.179094092 | YKL180W |
| 2.548906658 | YBL078C | −6.268339206 | YGR034W |
| 2.544977343 | YDR073W | −6.45149638 | YCL030C |
| 2.516739274 | YNL086W | −6.485115709 | YDL081C |
| 2.506545396 | YCL035C | −6.489343347 | YMR189W |
| 2.463693403 | YLR425W | −6.541348829 | YLR301W |
| 2.455193795 | YPR049C | −6.558891396 | YFR031C-A |
| 2.454956957 | YJL057C | −6.590790834 | YJR143C |
| 2.451635451 | YMR160W | −6.613092271 | YER003C |
| 2.450758689 | YDR505C | −6.653903754 | YKR013W |
| 2.441735494 | YOL025W | −6.658129975 | YPR102C |
| 2.441310938 | YCL026C-B | −6.670582649 | YKL081W |
| 2.439404524 | YLL015W | −6.680579383 | YEL054C |
| 2.437783756 | YKL124W | −6.690163145 | YIL018W |
| 2.408064062 | YOR215C | −6.724609452 | YLR175W |
| 2.395930167 | YHR156C | −6.749156344 | YHR183W |
| 2.382064524 | YER184C | −6.799042863 | YIL078W |
| 2.36850478 | YOL108C | −6.8589603 | YMR242C |
| 2.359236334 | YPL024W | −6.948221781 | YBL002W |
| 2.354787895 | YLL027W | −6.985707893 | YLR060W |
| 2.351229192 | YDL010W | −7.061239958 | YBL039C |
| 2.346099724 | YOR289W | −7.141447562 | YDL003W |
| 2.338141488 | YMR213W | −7.158692342 | YBR191W |
| 2.327917227 | YBR020W | −7.255600065 | YHR064C |
| 2.323750802 | YLR371W | −7.520388163 | YDL227C |
| 2.314375067 | YKL142W | −7.572943212 | YLR061W |
| 2.309296898 | YHL048C-A | −7.586083779 | YKL006W |
| 2.28846374 | YER020W | −7.69978181 | YGL031C |
| 2.277226641 | YNR010W | −7.857195334 | YHR068W |
| 2.276402092 | YAL017W | −7.922874579 | YJL190C |
| 2.274054982 | YOR034C | −7.957061147 | YOR096W |
| 2.255292947 | YNL027W | −7.967305573 | YMR142C |
| 2.250048462 | YCR037C | −8.0168959 | YLL045C |
| 2.249887206 | YOL077W-A | −8.075002589 | YDL083C |
| 2.237387532 | YFL026W | −8.163848295 | YER055C |
| 2.233757753 | YKR031C | −8.213778477 | YDR382W |
| 2.227193055 | YMR255W | −8.460656082 | YBL003C |
| 2.217109975 | YKR058W | −8.480176347 | YMR116C |
| 2.196570377 | YKL091C | −8.618883723 | YGR214W |
| 2.173266425 | YJR021C | −8.902481598 | YER131W |
| 2.166591863 | YOL032W | −8.940093866 | YLR029C |
| 2.166531772 | YML131W | −9.121021068 | YGL157W |
| 2.16578106 | YKL018C-A | −9.124518837 | YNL069C |
| 2.163525951 | YNR047W | −9.182577231 | YPL131W |
| 2.152884326 | YPL196W | −9.212522259 | YNL141W |
| 2.151098582 | YDL247W | −9.310985195 | YDL229W |
| 2.137807869 | YHR152W | −9.40611886 | YLR249W |
| 2.134304184 | YOR394W | −9.572157961 | YER011W |
| 2.133539487 | YML117W | −9.681520451 | YOR063W |
| 2.128884005 | YBL059W | −9.710581727 | YMR120C |
| 2.128037032 | YDL078C | −9.909492251 | YBR088C |
| 2.112238415 | YOL013C | −10.29407496 | YBR189W |
| 2.111910643 | YMR302C | −10.7021159 | YOL039W |
| 2.109107799 | YKL129C | −11.05850583 | YGR138C |
| 2.10685232 | YLL040C | −11.6727234 | YLR448W |
| 2.105535355 | YHR195W | −12.03724749 | YLR058C |
| 2.102684184 | YOL060C | −12.3701282 | YCL064C |
| 2.099084389 | YLR248W | −12.90807457 | YGL147C |
| 2.095704571 | YKL065C | −14.54478671 | YML056C |
| 2.078377363 | YIL071C | −15.40913929 | YER070W |
| 2.049999029 | YMR068W | −16.76005564 | YGL253W |
| 2.048391201 | YPL232W | −20.0249601 | YLL024C |
| 2.046875624 | YBR149W | −21.53016549 | YDR345C |
| 2.037837312 | YGR174C | −35.70367007 | YHR092C |
| 2.025042685 | YMR090W | | |
| 2.021551708 | YOR147W | | |
| 2.020189825 | YHR050W | | |
| 2.011934782 | YDR460W | | |
| 2.005401752 | YOR065W | | |

Promoters found to up-regulate the expression of genes to which they are operatively linked in response to conditions characteristic of the duodenum include those set out in the “Up” column of Table 4. Suitable promoters to be operatively linked to one or more genes associated with degradation of yeast cell wall components may be selected from this group, and preferred examples of such genes include those encoding cell wall component-degrading enzymes such as endochitinases (encoded by genes such as CTS1) or exo, 1-3β-glucanase (encoded by genes such as EXG1), as before.

Amongst the total number of gene promoters identified as being responsive to one or more conditions characteristic of the digestive tract, the inventors have discovered that certain promoters show particularly strong responses, whether in terms of decreasing or increasing expression of the genes to which they are operatively linked. These represent particularly preferred promoters for use in accordance with the present invention.

Particularly preferred promoters that decrease expression of a gene to which they are operatively linked in response to conditions characteristic of the digestive tract include the promoters of ANB1 (YJR047C); TIR1 (YER011W); and MF(ALPHA)2 (YGL089C); as well as those of YMR321C and YLR065C, both of which are genes of unknown function. Each of these promoters down-regulates gene expression in response to conditions found in both the stomach and the duodenum. Any of these promoters may be operatively linked to SRB1, to PCK1, or to both of these genes.

Particularly preferred promoters that increase the expression of a gene to which they are operatively linked in response to conditions characteristic of the digestive tract include the promoters of PCK1 (YKR097W); DDR2 (YOL052C-A); SPG4 (YMR107W); TMA10 (YLR3227CO); FBP1 (YLR377CO) and PUT4 (also designated YOR348C). Each of these promoters up-regulates gene expression in response to conditions found in both the stomach and the duodenum. However, PCK1, TMA10, DDR2, PUT4, each show particularly noticeable up-regulation of gene expression in response to conditions characteristic of the stomach, while FBP1 and SPG4 each show particularly increased up-regulation of gene expression in response to conditions characteristic of the duodenum. This difference in the conditions responded to may provide the basis for targeting of yeast cell lysis to different compartments of the digestive tract. Any of these promoters may be operatively linked to CTS1, to EXG1, or to both of these genes.

Those skilled in the art will readily be able to identify the promoters of the various genes identified by the inventors, either by reference to publicly available information (for example, publications or other sources identifying promoters of interest), by reference to the sequence upstream of the gene (where structures such as “TATA boxes” are frequently indicative of promoters) or through routine experimentation. The skilled person may, for example, investigate a region of between 100 bp, 500 bp and 1000 bp upstream of the open reading frame to identify that portion that exhibits promoter activity. Suitable techniques by which such investigations may be undertaken will be well known to those skilled in the art.

ANB1 (YJR047C) is a gene which encodes the A subunit of eukaryotic initiation factor elF-5A which is essential translation factor that promotes the formation of the first peptide bond. ANB1 is tightly repressed under aerobic conditions and is induced over 200-fold in cells grown under hypoxic conditions. Its transcription is regulated by Rox1 p, a repression factor which is induced only in aerobic cells.

TIR1 (YER011W) encodes a stress-response cell wall mannoprotein that is a member of a family of serine-alanine-rich proteins whose expression is down-regulated at acidic pH and, to a great extent, induced by cold shock, anaerobiosis and hypoxic conditions. TIR1 has, along with other genes, been suggested as necessary for the growth of yeast cells at low temperature.

Since the protein encoded by TIR1 is a mannoprotein component of the yeast cell wall, TIR1 may potentially be taken to constitute a gene associated with yeast cell wall integrity. In keeping with the requirement that the nucleic acid constructs used in the yeast cells of the invention (or in accordance with the third aspect of the invention) are “artificial”, it will be appreciated that when the promoter of TIR1 is used as a promoter responsive to conditions characteristic of the digestive tract it must be used to control a gene other than TIR1, and when TIR1 is used as a gene associated with yeast cell wall integrity, it must be operatively linked to a promoter other than that of TIR1.

MF(ALPHA)2 (YGL089C)) encodes a mating pheromone alpha factor, made by alpha cells, which determines interaction with cells of the opposite mating type (a) inducing cell cycle arrest, cell wall changes, morphological alterations and other responses leading to mating.

PCK1 (YKR097W) encodes phosphoenolpyruvate carboxykinase which catalyzes early reaction in carbohydrate biosynthesis. The gene product of PCK1 functions during gluconeogenesis to form phosphoenolpyruvate from oxaloacetate. The protein product is located in the cytosol and its transcription is repressed by glucose.

DDR2 (YOL052C-A) encodes a multistress protein tyrosine kinase whose expression is rapidly and strongly activated by a variety of xenobiotic agents and environmental or physiological stresses. Both SIRE (Stress Response Element) and the zinc-finger transcription factors, Msn2p and Msn4p are required for the multistress response of DDR2. The gene is transcribed at high level following exposure of yeast cells to DNA-damaging agents, e.g. methyl methane sulphonate (MMS) and ultraviolet light (UV), as well as to heat shock and other stresses. Genetic studies using gene deletion mutants have indicated that DDR2 is a non-essential gene; the deletion mutant has no obvious phenotype with respect to heat shock sensitivity.

SPG4 (YMR107W) encodes a protein required for survival at high temperature during stationary phase, and that is not required for growth on non-fermentable carbon sources. Its transcription is induced under aerobic conditions. It has also been reported that Spg4p plays a negative role in TOR signalling, which is up-regulated by rapamycin. The transcription of SPG4 is induced by the diauxic shift and is negatively regulated by Srb1/Psa10p complex together with other proteins involved in the morphological change that permits foraging for nutrients.

TMA10 (YLR3227CO) encodes protein of unknown function that associates with the ribosomes and cellular translation machinery. Previous studies suggested that Tma10p (the protein product of TMA10) might be involved in glycogen and energy reserve metabolism or that it might have a transferase function. Also the amino acid similarity of Tma10p to the Stf2 protein suggests that it is involved in the regulation of the mitochondrial F₁F₀-ATP synthase. It has been reported that TMA10 is down-regulated by cytotoxic stress (such as ethanol and NaCl), while it is up-regulated by genotoxic stress in coordination with STF2. In addition, its protein product was reported to have a putative cytoplasmic ribosome function and categorised as part of an assembly of protein complexes.

FBP1 (YLR377CO) encodes fructose-1,6-biphosphatase, which is a key enzyme in gluconeogenesis pathway that is required for glucose metabolism. Its expression is up-regulated when yeast is grown in medium containing a poor carbon source and it is rapidly down-regulated when glucose-starved cells are replenished with glucose.

PUT4 (YOR348C) encodes proline permease which is required for high-affinity transport of proline, alanine, and glycine. The protein product also transports the toxic proline analogue, azetidine-2-carboxylate (AzC). PUT4 is a nitrogen-regulated permease gene and so its transcription is repressed in the presence of yeast's preferred nitrogen sources such as ammonia, glutamine, and asparagine.

Specific Examples of Genes Associated with Yeast Cell Wall Integrity

A number of genes associated with yeast cell wall integrity have been mentioned in the preceding paragraphs. The selection of suitable genes associated with yeast cell wall integrity and relevant examples of such genes, will now be described in greater detail below.

Lysis of yeast cells of the present invention in the digestive tract occurs as a result of the physical and/or environmental conditions found within the digestive tract. These conditions cause the cell wall and cell membrane to rupture, and the cytoplasmic contents to be released. However, unlike naturally occurring yeasts, the yeast cells of the present invention are predisposed to such lysis, by virtue of the artificial nucleic acid construct that they incorporate. This construct comprises a gene associated with yeast cell wall integrity, which will typically be a gene encoding a product that is either required for biogenesis and/or maintenance of the yeast cell wall, or that is able to contribute to degradation of cell wall structural components. The gene is operatively linked to a promoter that responds to conditions characteristic of the digestive tract, and thereby regulates expression of the gene. Thus the selection of the promoter, which has been discussed in more detail above, enables targeting of the location in which lysis will occur, while the gene associated with yeast cell wall integrity provides the mechanism by which the cell is predisposed to lyse.

Genes associated with yeast cell wall integrity suitable for use in accordance with the present invention may be generally classified as either genes that contribute to cell wall biogenesis (expression of which should be decreased to promote cell lysis) and genes that contribute to cell wall degradation (expression of which should be increased to promote cell lysis).

Examples of genes that contribute to cell wall biogenesis suitable for incorporation in the artificial nucleic acid constructs include those encoding components of the yeast cell wall. Although the detailed composition of the yeast cell wall varies between species and growth conditions, it has a conserved core structure which has generally been well characterised. Common components include those selected from the group consisting of: mannans; glucans; and chitins. Various genes responsible for synthesis of these cell wall components are known, including CHS2 (involved in chitin synthesis); FKS1 (involved in glucan synthesis) and MNN9 (involved in mannan synthesis) that could be potentially of interest as further targets of cell wall integrity.

In addition to the genes that are responsible for the synthesis of the cell wall components themselves, a number of further genes have been identified that regulate the production and deposition of these cell wall components. These genes may also be suitable for use in the yeasts of the invention.

For example, PKC1 (the yeast homologue of protein kinase C) regulates the biosynthesis and assembly of major cell wall components by a PKC1-mediated signal transduction pathway. PKC1, in conjunction with Rho1p, regulates β-3 glucan synthetase. Yeast mutants lacking PKC1 can only grow in the presence of osmotic stabilisers, since loss of PKC1 function results in a cell-cycle-specific osmotic stability defect. PKC1 represents a preferred gene associated with cell wall integrity that may be used in accordance with the present invention.

SRB1 (also known as PSA1 or VIG9) encodes GDP-mannose pyrophosphorylase, the enzyme responsible for the production of a major substrate for all kinds of mannosylation reactions, involved in the biosynthesis of cell wall mannan (the complex of highly N- and O-glycosylated cell wall mannoproteins). A SRB1/PSA1 null mutation is lethal whereas a decrease in SRB1/PSA1 function (by inhibiting the expression of SRB1/PSA1) leads to defects in bud growth, bud site selection, and cell separation, in addition to increases in cell permeability and cell lysis. SRB1 is a preferred gene associated with cell wall integrity for use in accordance with the present invention.

When used in yeasts in accordance with the present invention, PKC1 and/or SRB1 should be operatively linked to promoters that decrease their expression in response to conditions characteristic of the digestive tract. In the event that both PKC1 and SRB1 are to have their expression down-regulated, these genes may be operatively linked to the same promoter, or to different promoters each capable of down-regulating gene expression in response to conditions characteristic of the digestive tract. It may be preferred that one or both of these genes is operatively linked to a promoter that down-regulates gene expression in response to conditions found in the stomach and in response to conditions found in the small intestine.

There are a number of genes that encode agents that contribute to degradation of cell wall components. These include genes encoding enzymes that digest one or more cell wall components. Examples of such enzymes include endochitinases; glucanases; and mannanases.

The inventors have identified two enzymes capable of degrading cell wall components that represent particularly preferred examples of this class of genes suitable for use in accordance with the present invention.

The first of these is the endochitinase encoded by the gene CTS1 whose transcriptional activation during the G1 phase of the cell cycle is mediated by transcription factor Ace2p.

The enzyme Cts1p is required for cell separation after mitosis.

The second preferred enzyme is the major exo, 1-3β-glucanase encoded by EXG1. This enzyme is able to break down the cell wall glucan component.

It will be appreciated that, when used in accordance with the present invention, CTS1 and/or EXG1, or other such genes encoding agents capable of degrading yeast cell wall components, should be operatively linked to one or more promoters that increase their expression in response to conditions characteristic of the digestive tract. It may be preferred that one or both of these genes is operatively linked to a promoter that increases gene expression in response to conditions found in the stomach and in response to conditions found in the small intestine.

Preferred yeast cells in accordance with the invention may comprise an artificial nucleic acid construct incorporating at least one gene associated with yeast cell wall integrity selected from the group consisting of: PKC1; SRB1; CTS1 and EXG1; operatively linked to at least one promoter responsive to conditions characteristic of the digestive tract selected from the group consisting of the promoters of: ANB1; TIR1; MF(ALPHA)2; PCK1; DDR2; SPG4; TMA10; FBP1 and PUT4.

In a particularly preferred embodiment, yeast cells in accordance with the invention may comprise an artificial nucleic acid construct incorporating at least one gene associated with yeast cell wall integrity selected from the group consisting of: PKC1 and SRB1; operatively linked to at least one promoter responsive to conditions characteristic of the digestive tract, selected from the group consisting of the promoters of: ANB1; TIR1 and MF(ALPHA)2.

In a further particularly preferred embodiment, yeast cells in accordance with the invention may comprise an artificial nucleic acid construct incorporating at least one gene associated with yeast cell wall integrity selected from the group consisting of: CTS1 and EXG1; operatively linked to at least one promoter responsive to conditions characteristic of the digestive tract, selected from the group consisting of: PCK1; DDR2; SPG4; TMA10; FBP1 and PUT4.

A yeast cell of the invention may comprise an artificial nucleic acid construct incorporating a first gene encoding a yeast cell wall component operatively linked to a first promoter that decreases expression of the first gene in response to conditions characteristic of the digestive tract, and a second gene encoding an agent capable of degrading a yeast cell wall component operatively linked to a second promoter that increases expression of the second gene in response to conditions characteristic of the digestive tract.

### Agents of Interest

The yeast cells of the invention may be used for the delivery of a wide range of agents of interest. Generally, suitable agents of interest that may be delivered by the yeast cells of the invention include any compounds that can be encapsulated within yeast.

It may be preferred that the agents of interest are expressed by the yeast cells, and so encapsulated in the yeast cells in this manner. This offers notable advantages in that a single vehicle is thus used to produce and deliver the agent of interest, thereby cutting out the costly post-production processes of extraction, purification, preservation and encapsulation. Agents of interest suitable for use in connection with this embodiment will typically be gene products, such as proteins, peptides or nucleic acids. Accordingly, it is a preferred embodiment of the invention that the yeast cells further comprise a nucleic acid sequence encoding an agent of interest. The agent of interest may be expressible by the cell. Suitable agents may be therapeutic agents (as considered further below).

The yeast cells of the invention are capable of expressing and delivering a wide range of therapeutic agents and vaccines, including those produced in the form of large protein complexes. They provide means by which therapeutic molecules that are normally degraded by gastric conditions may be produced and delivered, and obviate the need for downstream purification process.

Yeast cells have the capacity to reversibly open up junctions between cells lining the digestive tract, and this may enhance the uptake of agents via the tract. Thus the yeast cells of the invention may serve to promote uptake of an agent of interest that they deliver. The uptake of peptide and protein agents through the digestive tract can be further increased by their modification to incorporate absorption enhancers. Examples of such enhancers include: surfactants; detergents; bile salts; additional amino acids and/or fatty acids. Protein or peptide agents of interest to be delivered using the yeast cells of the invention may be modified using one of more of these enhancers in order to improve uptake efficiency.

Agents of interest that may be expressed, encapsulated and subsequently delivered by the yeast cells of the invention include the expression products of naturally occurring yeast genes, the expression products of heterologous genes, and the expression products of synthetic gene constructs. Generally it may be preferred to use native yeast promoters to control such expression, but other non-yeast promoters may also be used.

Suitable promoters may be selected with reference to the purpose to be achieved. It may, for example, be wished to employ a promoter that maximises production of an agent of interest prior to lysis. Alternatively, it may be wished to employ a promoter that results in production of the agent of interest only in response to specific growth conditions (such as growth phase or rate of the yeast cells, their respiratory capacity or biomass yield).

It may be wished to use constitutive and non-regulatable promoters to control the expression of agents of interest. Such promoters may be expected to achieve constitutive high levels of expression. The pPGK1 promoter is an example of such a promoter. PGK1 encodes phosphoglycerate kinase1, an enzyme in the glycolytic pathway. The use of this promoter in yeast cells of the invention will lead to continuous production of the agent of interest.

If expression of an agent of interest by the yeast cells will affect other production factors such as biomass (as in batch culture) it may be preferred to use promoters that work later in the batch growth curve such as pSML1 and pSSA3. The pSML1 promoter's level of expression fluctuates during the cell cycle, being lowest at the S phase. The pSSA3 promoter determines low expression levels under optimal growth conditions and dramatically increases levels in response to heat shock. Both promoters have been shown to significantly induce transcription levels of their respective adjacent genes at the late stages of growth in batch cultures.

Use of the HXT5 promoter to control the expression of genes encoding an agent of interest may be a preferred choice since this promoter doesn't interfere with the normal growth of the yeast cells, yet still exhibits high expression efficiency. pHXT5 is regulated by growth phase of the yeast cells as expression of HXT5 is at its highest during slow growth caused by increased osmolarity, presence of non-fermentable carbon sources, increased temperature or during stationary phase.

Other previously published, or commercially available systems for the regulation of genes encoding agents of interest may also be used in accordance with the invention. Merely by way of example, Novozyme have developed a proprietary Saccharomyces cerevisiae based expression system for high yielding, animal-free protein production of proteins, and this system may be used to regulate expression of suitable agents of interest.

Although the preceding paragraphs describe a number of preferred embodiments of the invention, the applications of the invention are not limited to the delivery of agents expressed in the yeast cells.

Agents of interest may be of interest for a number of reasons, including for purposes of experimental research. However, it may generally be preferred that agents of interest to be delivered by the yeast cells of the invention are therapeutic agents.

### Therapeutic Agents

Yeast cells of the invention represent suitable vehicles by which therapeutic agents may be provided to subjects in need thereof. This use is the basis of the methods of the invention, which are suitable for use in methods of treatment where an agent of interest such as a therapeutic agent, is to be provided to a subject. The following passages will describe various therapeutic agents that may be provided to a subject by the yeast cells of the invention, or the methods of the invention.

As set out above, a therapeutic agent in accordance with the present invention may be any agent suitable for use in the prevention and/or treatment of a disease or other condition detrimental to a subject. It may generally be preferred that the therapeutic agents comprise proteins or peptides. Such protein or peptide therapeutic agents may preferably be expressed by the yeast cells of the invention. Preferred embodiments for use in such expression are described further below.

Suitable therapeutic agents that may be provided using the yeast cells or methods of the invention include peptide growth factors; hormones; immunosuppressants; anti-retrovirals; anti-coagulants; antibodies; antibiotics; vaccines; and other peptides and proteins with potential therapeutic action. Accordingly, yeast cells of the invention may further comprise nucleic acid sequences encoding a therapeutic agent selected from the group consisting of: peptide growth factors; hormones; immunosuppressants; anti-retrovirals; anti-coagulants; antibodies; antibiotics and vaccines.

Merely by way of example, the inventors believe that the yeast cells or methods of the invention may be used to provide one or more agents independently selected from the group consisting of: insulin; vancomycin; oxytocin; cyclosporine; enfuvirtide; and eptifibatide to subjects in need thereof.

Appropriate agents, such as those considered above, may be used prophylactically, to prevent a disease, infection or the like, or in the treatment of existing diseases, infections or disorders.

The yeast cells of the invention may be of use in the delivery of agents of interest, and particularly therapeutic agents, to human and non-human animal subjects. For example, the use of the yeast cells of the invention for the provision of vaccines to domestic or farm animals is of significant advantage, since it does away with the need for administration by injection (thus reducing the veterinary equipment and expertise required) and allows large numbers of animals to be provided with vaccines at the same time. Yeast cells in accordance with the invention may be mixed with foodstuffs or drinks to allow their oral administration.

The inventors believe that the use of yeast cells of the invention to administer agents of interest may expand the range of agents capable of therapeutic application. Since the agents of interest are encapsulated (and thus shielded from many conditions in the digestive tract that may otherwise cause their breakdown and loss of function) they may allow agents that are otherwise sensitive to degradation in the digestive tract to be used in a manner that was not previously possible.

### Experimental Results

### (1) Selection of Promoters Producing the Largest Decrease in Cell Wall Gene Expression in Response to Conditions Characteristic of the Digestive Tract

The effect of gastric and duodenal conditions on transcription levels of the genes ANB1 (YJR047C), TIR1 (YER011W), and MF (ALPHA)2 (YGL089C) are investigated to identify those genes having promoters that most markedly decrease transcription levels in response to conditions characteristic of the digestive tract. These genes have all been shown to manifest large decreases in their transcript abundances when investigated using microarray studies. Their promoters are to be used to regulate SRB1/PSA1 and PKC1 in vivo. The temporal pattern of changes in the transcription levels of these genes are determined in response to a time course exposure to models of gastric and duodenal conditions. Changes in transcript abundance revealed by microarray analysis are confirmed using methods such as Northern analysis and qRT-PCR.

The preferred promoters identified by this study are referred to as PdX and PdY below.

### (2) Selection of Promoters Producing the Largest Increase in Cell Wall Degradation Proteins in Response to Conditions Characteristic of the Digestive Tract

The effect of gastric and duodenal conditions on transcription levels of the genes PCK1 (YKR097W), DDR2 (YOL052C-A), SPG4 (YMR107W), TMA10 (YLR327C), FBP1 (YLR377C), and PUT4 (YOR348C) are also investigated to identify those genes having promoters that most markedly increase transcription levels in response to conditions characteristic of the digestive tract. These genes have all been shown to manifest large increases in their transcript abundances when investigated using microarray studies. Their promoters are to be used to regulate the transcription of genes encoding CTS1 and EXG1 in vivo. The temporal pattern of changes in the transcription levels of these genes are determined in response to a time course exposure to models of gastric and duodenal conditions. Changes in transcript abundance revealed by microarray analysis are confirmed using methods such as Northern analysis and qRT-PCR.

The preferred promoters identified by this study are referred to as PuX and PuY below.

### (3) Creation of Mutant Strains in which Cell Wall Genes are Down Regulated

The transcription of SRB1 and PKC1 is regulated by fusing the corresponding open reading frames (ORFs) of these genes to the preferred promoters identified in stage (1). As a result of this a yeast strain is constructed in which the endogenous promoters of SRB1 and PKC1 had been replaced by the preferred promoters down-regulating gene expression in response to conditions characteristic of the digestive tract. This study will confirm that transcription of SRB1 and PKC1 under the control of these promoters responsive to conditions characteristic of the digestive tract leads to lysis of the yeast cells in response to conditions found in the human stomach and duodenum. A parental strain of yeast from which CHS3 is deleted will be used for this purpose, and the strains shown below generated.

| | | | |
|---|---|---|---|
| PdX-PKC1 | PdY-PKC1 | PdY-PKC1 | PdX-PKC1 |
| PdX-SRB1 | PdY-SRB1 | PdX-SRB1 | PdY-SRB1 |

The levels of PKC1 and SRB1 transcripts' down-regulation upon gastric and duodenal conditions are determined by qRT-PCR, and those of the corresponding proteins by Western blot analysis using custom-produced polyclonal antibodies. The lysis ability of the respective mutants are evaluated by diagnostic tests. The two best performing strains identified as a result of stage 2 (referred to as A and B below) are investigated further.

### (4) Creation of Mutant Strains in which Cell Wall Degradation Genes are Up Regulated

Following on from study 3 above, the transcription of CTS1 and EXG1 is also regulated by fusing the corresponding ORFs to the promoters PuX and PuY identified in stage 2. This study illustrates that replacement of the endogenous promoters of CTS1 and EXG1 with those of PuX and PuY enhances and accelerates lysis of the yeast cells upon exposure to conditions characteristic of the human digestive tract. Eight yeast strains based on the use of both strains A and B generated in (3) are constructed.

| Strain A | Strain B |
|---|---|
| PuX-CTS1; PuX-EXG1 | PuX-CTS1; PuX-EXG1 |
| PuY-CTS1; PuY-EXG1 | PuY-CTS1; PuY-EXG1 |
| PuY-CTS1; PuX-EXG1; | PuY-CTS1; PuX-EXG1; |
| PuX-CTS1; PuY-EXG1 | PuX-CTS1; PuY-EXG1 |

The lysis ability of these strains are compared using diagnostic lysis tests. The two best performing new strains referred to as C and D are then tested in a recognised computer controlled model system of the gut developed at the Norwich Institute of Food Research (and described in patent application WO 2007/010238, the disclosure of which is hereby incorporated by reference).

### (5) Investigation of Candidate Strains for Lysis Ability In Vitro

Strains are tested in vitro using several methods used routinely in the inventor's laboratory. These studies will examine yeast cell lysis under various conditions, including simulated stomach and gut environments. Lysis will be monitored over a specified time course and unlysed yeast cells will be collected to monitor the efficacy of the gene targeted lysis and to calculate percentage of yeast cell lysis versus an untreated control.

### (6) Investigation of Candidate Strains for Lysis Ability Ex Vivo

Modified strains will also undergo similar trials in the ex vivo gastrointestinal tract model developed at the Institute of Food Research. Cell lysis at various stages during simulated digestion will be monitored. Results will give an accurate indication as to how efficient lysis will be in vivo.

1. A yeast cell comprising an artificial nucleic acid construct incorporating a gene associated with yeast cell wall integrity operatively linked to a promoter responsive to conditions characteristic of the digestive tract.

2. A yeast cell according to claim 1, wherein the gene associated with yeast cell wall integrity is a gene expression of which increases accumulation of yeast cell wall components; and the promoter is a promoter that down-regulates gene expression in response to conditions characteristic of the digestive tract.

3. A yeast cell according to claim 1, wherein the gene is a gene expression of which increases the degradation of cell wall components; and the promoter is a promoter that up-regulates gene expression in response to conditions characteristic of the digestive tract.

4. A yeast cell according to claim 1, wherein the gene encodes an enzyme that causes the degradation of yeast cell wall components.

5. A yeast cell according to claim 1, wherein the promoter is selected from the group consisting of: promoters responsive to conditions characteristic of the oral cavity; promoters responsive to conditions characteristic of the oesophagus; promoters responsive to conditions characteristic of the stomach; promoters responsive to conditions characteristic of the small intestine; promoters responsive to conditions characteristic of the large intestine; and promoters responsive to conditions characteristic of the rectum.

6. A yeast cell according to claim 1, wherein the promoter is responsive to conditions characteristic of the stomach selected from the group consisting of: very low pH; high temperature; the presence of digestive enzymes; and the presence of nutrients.

7. A yeast cell according to claim 1, wherein the promoter is responsive to conditions characteristic of the duodenum selected from the group consisting of: slightly reduced pH; high temperature; the presence of digestive enzymes; the presence of calcium carbonate; and the presence of bile salts.

8. A yeast cell according to claim 1, wherein the promoter is selected from the group of genes set out in Table 1.

9. A yeast cell according to claim 1, wherein the promoter is selected from the group of genes set out in Table 2.

10. A yeast cell according to claim 1, wherein the gene associated with yeast cell wall integrity is selected from the group consisting of: PKC1; SRB1; CTS1 and EXG1.

11. A yeast cell according to claim 1, wherein promoter responsive to conditions characteristic of the digestive tract selected from the group consisting of the promoters of: ANB1; TIR1; MF(ALPHA)2; PCK1; DDR2; SPG4; TMA10; FBP1 and PUT4.

12. A yeast cell according to claim 1, comprising an artificial nucleic acid construct incorporating at least one gene associated with yeast cell wall integrity selected from the group consisting of: PKC1 and SRB1; operatively linked to at least one promoter responsive to conditions characteristic of the digestive tract, selected from the group consisting of the promoters of: ANB1, TIR1 and MF(ALPHA)2.

13. A yeast cell according to claim 1, comprising an artificial nucleic acid construct incorporating at least one gene associated with yeast cell wall integrity selected from the group consisting of: CTS1 and EXG1; operatively linked to at least one promoter responsive to conditions characteristic of the digestive tract, selected from the group consisting of: PCK1; DDR2; SPG4; TMA10; FBP1 and PUT4.

14. A yeast cell according to claim 1, further comprising a nucleic acid sequence encoding an agent of interest.

15. A yeast cell according to claim 1, further comprising a nucleic acid sequence encoding a therapeutic agent.

16. A yeast cell according to claim 1, further comprising a nucleic acid sequence encoding a therapeutic agent selected from the group consisting of: peptide growth factors; hormones; immunosuppressants; anti-retrovirals; anti-coagulants; antibodies; antibiotics and vaccines.

17. A yeast cell according to claim 1, wherein the yeast cell is of a species selected from the group consisting of: S. cerevisiae and S. boulardii .

18. A yeast cell according to claim 1, comprising an artificial nucleic acid construct incorporating a first gene encoding a yeast cell wall component operatively linked to a first promoter that decreases expression of the first gene in response to conditions characteristic of the digestive tract, and a second gene encoding an agent capable of degrading a yeast cell wall component operatively linked to a second promoter that increases expression of the second gene in response to conditions characteristic of the digestive tract.

19. A method of providing an agent of interest to a subject in need thereof, the method comprising administering to the digestive tract of the subject a yeast cell comprising an artificial nucleic acid construct comprising a gene associated with yeast cell wall integrity, operatively linked to a promoter responsive to conditions characteristic of the digestive tract, wherein the yeast cell further comprises an amount of an agent of interest encapsulated within said yeast cell.

20. A method according to claim 19, wherein the therapeutic agent is selected from the group consisting of: peptide growth factors; hormones; immuno-suppressants; anti-retrovirals; anti-coagulants; antibodies; antibiotics and vaccines.

21. A method according to claim 19, wherein the yeast cell is administered orally.

22. A pharmaceutical composition comprising a yeast cell incorporating an artificial nucleic acid construct incorporating a gene associated with yeast cell wall integrity operatively linked to a promoter responsive to conditions characteristic of the digestive tract.

23. An artificial nucleic acid construct comprising a gene associated with yeast cell wall integrity operatively linked to a promoter responsive to conditions characteristic of the digestive tract.

24. A method of producing a yeast cell adapted for targeted lysis in the digestive tract, the method comprising introducing an artificial nucleic acid construct, incorporating a gene associated with yeast cell wall integrity operatively linked to a promoter responsive to conditions characteristic of the digestive tract, into a yeast cell, such that the artificial nucleic acid construct is expressible in the yeast cell.
